(19) United States
(12) Patent Application Publication
(10) Pub. No.: US 2008/0155713 A1
(43) Pub. Date: Jun. 26, 2008

(54) Lipid Metabolic and Signalling Pathways in the Epidermis

(75) Inventors: Alexander Yephremov, Koln (DE); Heinz Saedler, Koln (DE); Klaus-Gunther TietJen, Langenfeld (DE); Sandra Trenkamp, Berlin (DE); Lukas Schreiber, Alfter (DE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignees: BAYER CROPSCIENCE GMBH, Frankfurt (DE); MAXPLANCK-GESELLSCHAFT ZUR FÖRDERUNG DER WISSENSCH, Munchen (DE)

(21) Appl. No.: 11/659,867

(22) PCT Filed: Aug. 9, 2005
(86) PCT No.: PCT/EP05/08640

§ 371 Date: Oct. 2, 2007

(30) Foreign Application Priority Data
Aug. 9, 2004 (EP) 04018824.5

(51) Int. Cl. A01H 1/00; A01H 5/00; C12N 5/14; C07C 51/00; C07H 21/04

(52) U.S. Cl. 800/281; 800/298; 435/419; 562/400; 536/23.6

(57) ABSTRACT

The present invention relates to the field of genes isolated from Arabidopsis that code for enzymes that are involved in -oxidation of very long chain fatty acids, particularly, in conversion of ω-hydroxy fatty acid to fatty aldehyde. The invention also includes methods of producing transgenic plants with an altered fatty acid composition. Furthermore, the present invention relates to assays making use of these enzymes as herbicide targets to identify inhibitors of said enzymes, thereby providing herbicidally active compounds.

The present invention relates to the field of genes isolated from Arabidopsis that code for enzymes that are involved in ω-oxidation of very long chain fatty acids, particularly, in conversion of ω-hydroxy fatty acid to fatty aldehyde. The invention also includes methods of producing transgenic plants with an altered fatty acid composition. Furthermore, the present invention relates to assays making use of these enzymes as herbicide targets to identify inhibitors of said enzymes, thereby providing herbicidally active compounds.

Very long-chain fatty acids (VLCFA) could be metabolised by three oxidation pathways targeting a carbon in the α-, β- and ω-position in the chain (Bremer and Osmundsen, 1984). The majority of fatty acids are channelled into the β-oxidation pathway in peroxisomes that results in progressive shortening of fatty acids. The endoplasmic reticulum-localised ω-oxidation is generally considered as a minor pathway, which can be activated in case β-oxidation fails to proceed normally. It should be pointed out, however, some fatty acid derivatives are predominantly inactivated through ω-oxidation pathway (e.g. β-methyl-heptadecanoic acid in the heart (Fink et al., 1990), leukotriene B4 (LTB4) in human neutrophils (Sumimoto and Minakami, 1990) and rat hepatocytes (Wheelan et al., 1999)).

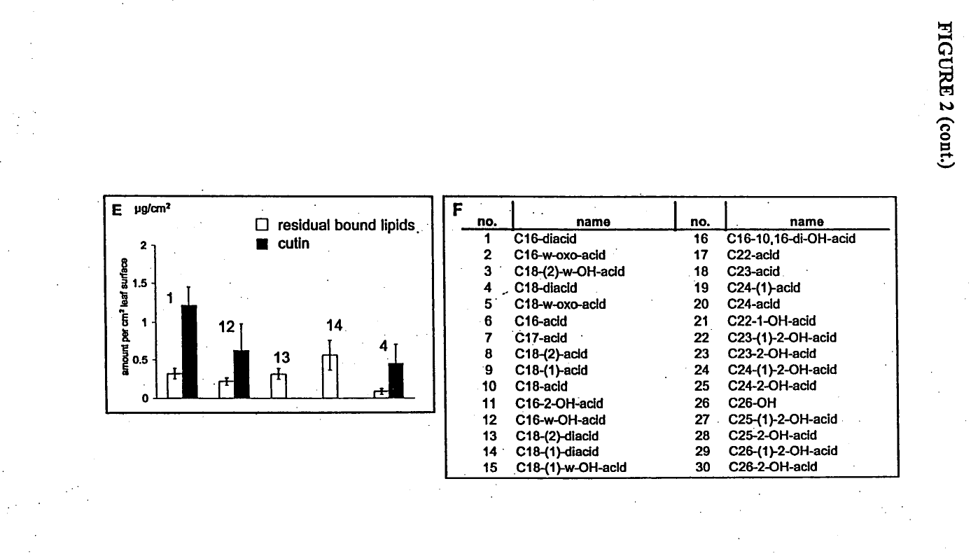

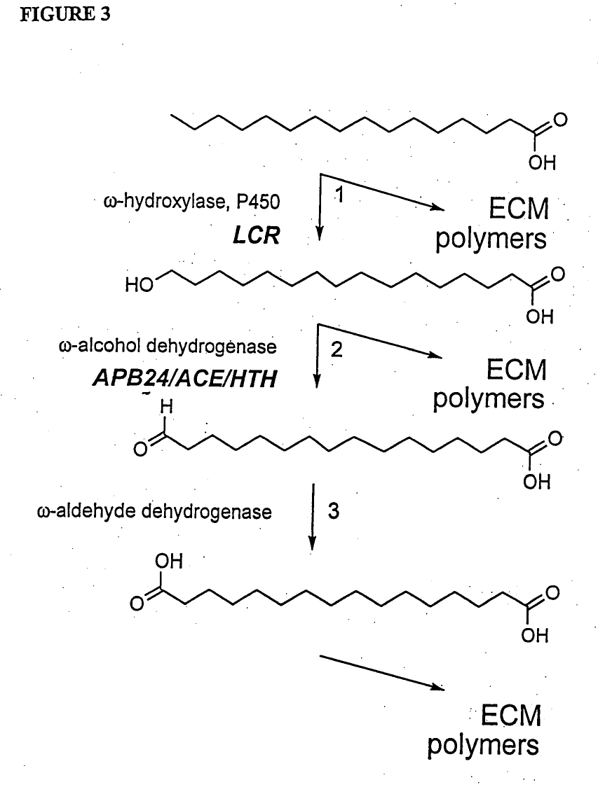

In contrast to the β-oxidation, the ω-oxidation of VLCFAs does not require their activation with coenzyme A (CoA). The ω-oxidation proceeds in three steps (FIG. 3). Firstly, the ω-carbon of VLCFA is oxidised to an alcohol, then to an aldehyde and at last to a carboxylic acid. The resulting product is a very long chain α-,ω-dicarboxylic fatty acid, which has carboxyl groups on both α- and ω-ends. Products of ω-oxidation can be activated into very long chain dicarboxylyl-CoAs esters by dicarboxylyl-CoA synthetase (Vamecq et al., 1985). Therefore, when the ω-oxidation is coupled to the β-oxidation, very long chain α-,ω-dicarboxylic fatty acids may be shortened and effectively metabolised. Accordingly, the stimulation of ω-oxidation is linked to induction of peroxisomal proliferation.

The first step of ω-oxidation (FIG. 3), is catalysed by ω-hydroxylases, which belong to cytochrome P450 gene family 4 in mammals, to family 52 in several Candida species (Nelson et al., 1996) and to family 86 in plants (Benveniste et al., 1998; Werck-Reichhart et al., 2001). At the next two steps (FIG. 3), the very long chain fatty alcohol is oxidised to α-,ω-dicarboxylic fatty acid by action of fatty alcohol:NAD oxidoreductase complex (Lee, 1979; Rizzo et al., 1987), which consists of separate proteins: fatty alcohol dehydrogenase (ADH, EC 1.1.1.1) catalysing oxidation of fatty alcohol to fatty aldehyde, and fatty aldehyde dehydrogenase (AldDH, EC 1.2.1.5), catalysing oxidation of fatty aldehyde to α-,ω-dicarboxylic fatty acid (Ichihara et al., 1986a; Ichihara et al., 1986b). ADH and AldDH use reduced NADH and oxidised NAD⁺ (nicotinamide adenine dinucleotide) as cofactors, respectively.

In animals, products of ω-oxidation are considered as intermediate metabolites, elevated levels of which in urine (dicarboxylic aciduria) could be used as an indicator of a fatty acid metabolism disorder. In plants, α-,ω-dicarboxylic fatty acids are structural materials that are found mainly in the protective coverings formed by cutin and suberin deposited in the cell walls. Very long chain α-,ω-dicarboxylic fatty acids represent aliphatics nearly unique to suberin (Matzke and Riederer, 1991). Although they were reported, for example, in cutin of Solanum tuberosum, Spinacia oleracea and species of Brassicacea, they generally comprise a minor proportion of the total cutin monomers (reviewed in (Holloway, 1982)). In contrast, α-,ω-dicarboxylic fatty acids belong to major structural components of suberin comprising up to 33% of monomers (Bernards, 2002; Holloway, 1983).

Suberin deposited in the cell walls in the root endodermis forms a waterproof cylinder called the Casparian strip. It is also detectable in aerial organs in the cells surrounding vascular bundles (bundle sheath). Suberin is a major structural component, which accumulates following wounding to protect plant against pathogens and water loss. Although being an important defence response, formation of suberin barrier may result in premature wilting in cut flowers because stems are no longer able to take up water.

Biochemical evidence for the presence of wound-induced ω-hydroxyfatty acid dehydrogenase required for biosynthesis of α-,ω-dicarboxylic fatty acids in suberin has been provided (Agrawal and Kolattukudy, 1978a; Agrawal and Kolattukudy, 1978b). The enzyme has been purified 600-fold from wound-healing potato (Solanum tuberosum L.) tuber disks to near homogeneity, and its molecular weight has been estimated to be about 31.000 Da.

The nucleotide and amino acid sequences of this enzyme remained unknown. No other nucleic acid sequences for ω-hydroxyfatty acid dehydrogenases that are involved in the biosynthesis of extracellular matrix polymers in plants have been provided.

The only genes that encode for very long chain fatty alcohol dehydrogenases involved in ω-oxidation have been isolated from Candida cloacae and related yeast species capable of growing on alkanes and fatty acids as sole carbon sources (Vanhanen et al., 2000). Physiological function of these enzymes involved in a degradative pathway of fatty acids is apparently quite different from enzymes involved in suberin and cutin production in plants. In databases, plant proteins could be found that show sequence similarities to fatty alcohol dehydrogenases isolated from Candida (Vanhanen et al., 2000). These proteins, however, are sufficiently distinct from the protein described in this invention. The recent publications describing molecular isolation of the ADHESION OF CALYX EDGES (ACE) or HOTHEAD (HTH) gene of Arabidopsis (Araki et al., 1998; Araki and Nakatani-Goto, 1999; Krolikowski et al., 2003) that is identical to the APB24 gene described in this invention, did not contain an indication or an evidence with regard to function of the protein.

The problem to be solved by the present invention is to provide a transgenic plant having a changed lipid composition as compared to a corresponding non-transgenic wild type plant. It is a further aim of the present invention to provide a transgenic plant having a changed lipid composition for use in the production of biopolymers on fatty acid basis. It is a further aim of the present invention to provide a transgenic plant having a changed lipid composition for use in the production of therapeutically active agents, cosmetic or pharmaceutical compositions. It is yet a further aim of the present invention to provide a method for selecting inhibitory compounds.

The problem is solved by the subject-matter defined in the claims.

The present invention is further illustrated by the following figures.

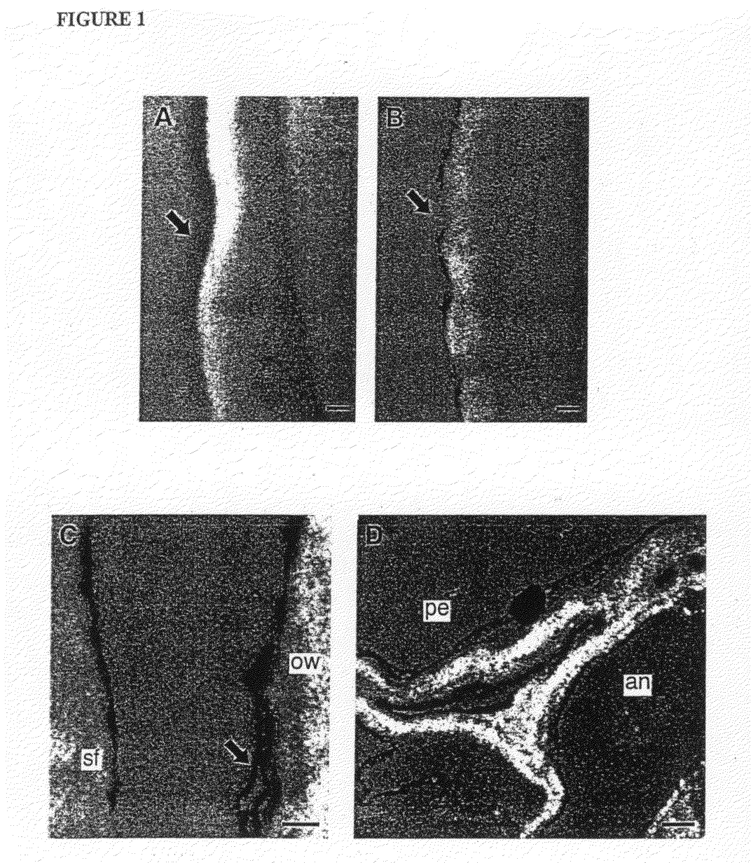

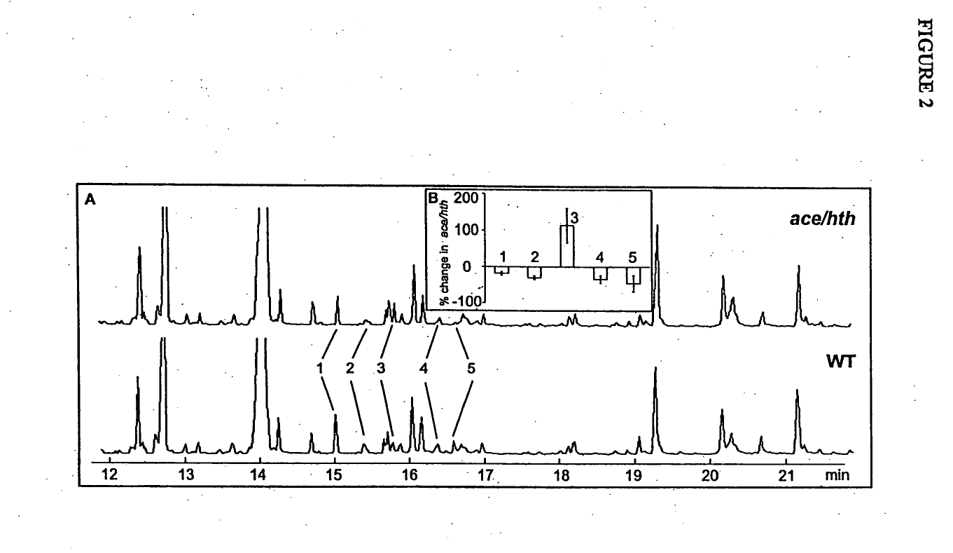

FIG. 1. Cuticular defects associated with the loss of the APB24 function.

To illustrate the mutant phenotype, floral organs, in which the APB24 is strongly expressed, have been inspected with transmission electron microscopy. Scale are 0.1 μm for (A to C), 0.5 μm for (D). (A to D) Cuticle proper (arrows) of septum is seen on electron micrographs as a dark layer above the light cell wall. In mutants, it may be discontinuous (B) or multilayered (C) compared to wild type (A); sf—stamen filament, ow—ovary wall. (D) Cell wall fusions between anther an and petal pe.

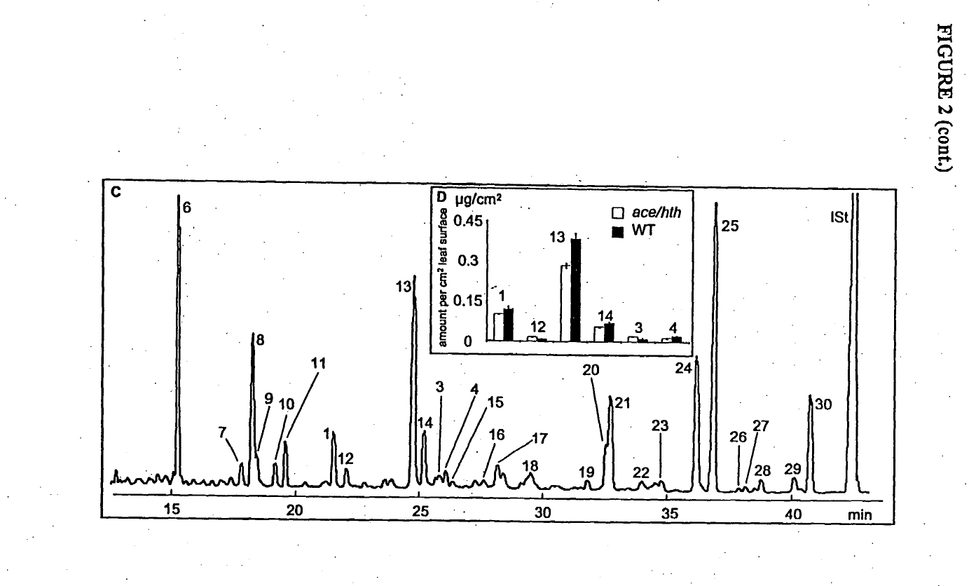

FIG. 2. Function of APB24 in plants in biosynthesis of fatty acids. Fatty acid metabolites have been analysed using GC-MS. (A) Comparative metabolic profiling of total lipids extracted from APB24 mutant and wild type inflorescences. (B) Five metabolites (1-5), the accumulation of which is affected in the APB24 mutant, are shown as mean (±SE) percentage changes (n=3). (C) Metabolic profiling of residual bound lipids extracted from wild type mature leaves. (D) Comparative analysis of ω-fatty acid hydroxylation in APB24 and wild type leaves (n=6 for APB24; n=5 for wild type). Accumulated metabolites are shown as μg per square cm. (E) Comparative analysis of leaf residual bound lipids and cutin (n=3) in the close relative of Arabidopsis broccoli. Note that unsaturated α-,ω-dicarboxylic fatty acids are not found in cutin. They may be associated with other components of the epidermal cell wall. (F) Peak identities in (A to E).

FIG. 3. Role of APB24 in the fatty acid omega-oxidation pathway in plants.

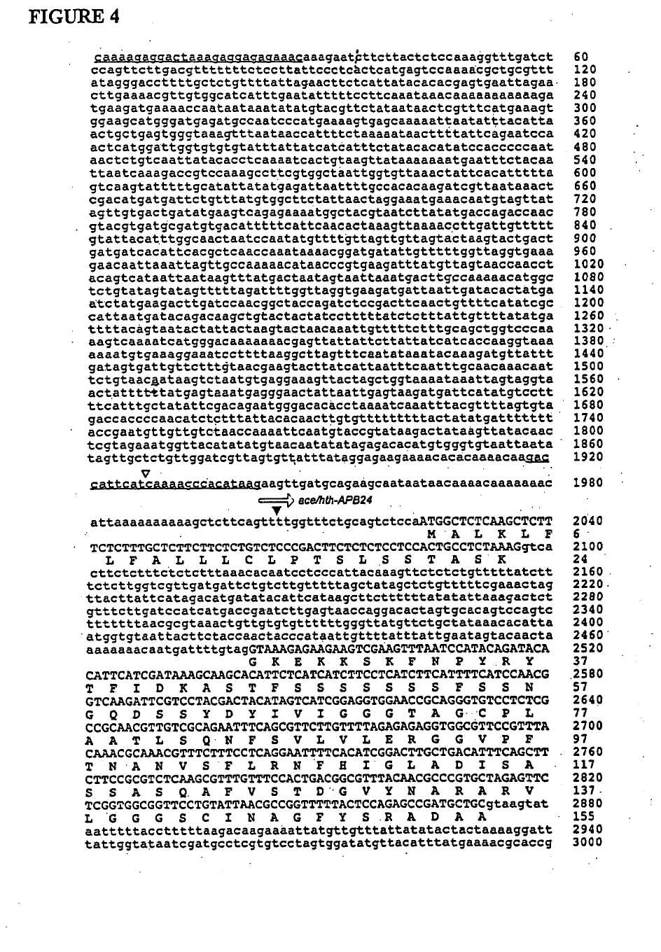

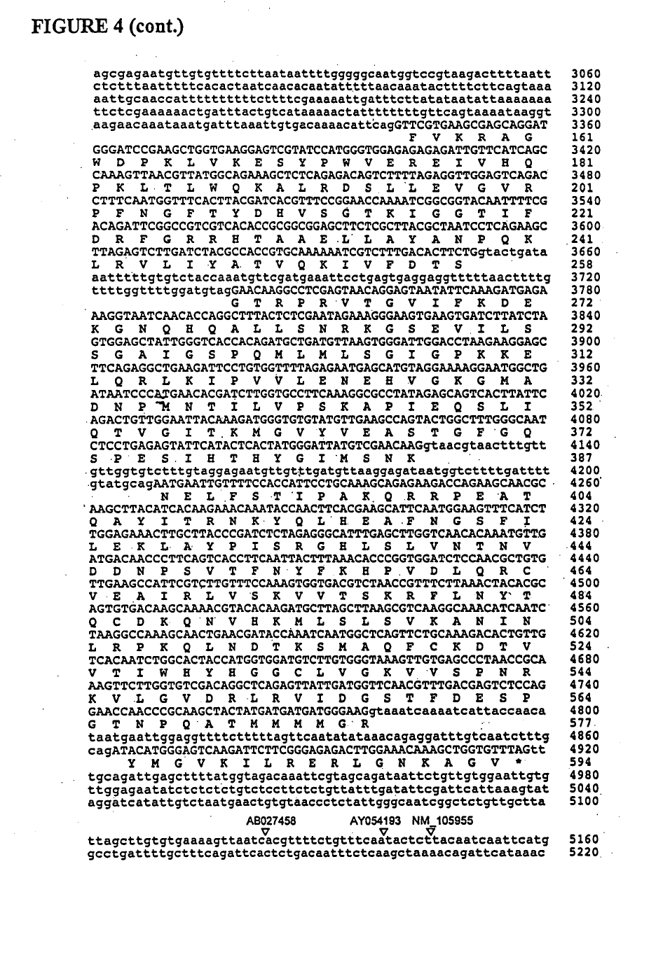

FIG. 4. Genomic organization of the APB24 gene This figure sets forth the DNA and amino acid sequences of the APB24 gene, including its promoter. The sequences shown here were obtained from Genbank clones (Genbank accession numbers AC008017, AB027458, AY054193, NM105955) and from sequencing of cDNA and promoter clones. Coding sequences are shown in uppercase letters, and 5′ and 3′ non-coding sequences and introns are shown in lowercase letters. The transcription start sites and polyadenylation sites, as determined from full size cDNA clones (Genbank accession numbers AB027458, AY054193, NM_{—}105955), are indicated by the inverted open triangles. Positions of the transposon insertion is indicated by the inverted black triangles and its orientation is shown by an arrow. Primer sequences used for isolation of the promoter of the APB24 gene are underlined. Numbers to the right represent nucleotide and amino acid positions.

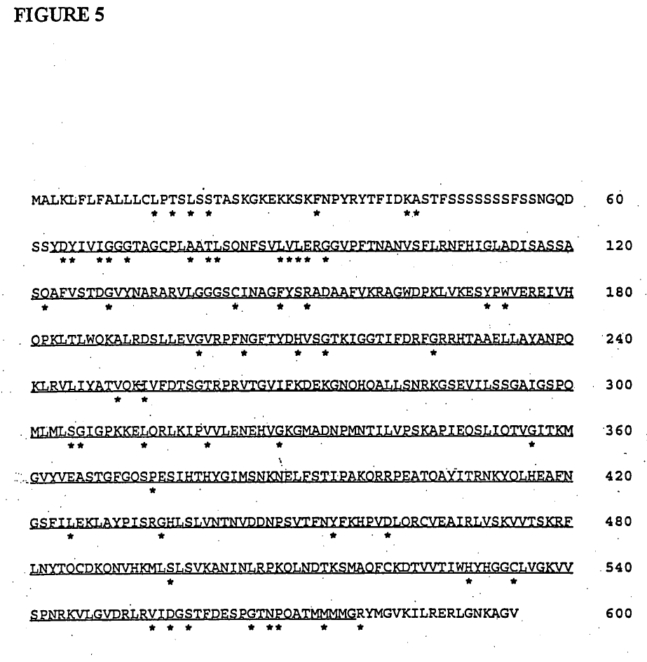

FIG. 5. (=SEQ ID NO:2) Amino acid sequence of the APB24 protein Amino acid positions from 63 to 580 corresponding to the fragment that exhibits the highest degree of sequence similarity to published glucose-methanol-choline (GMC) oxidoreductases (Cavener, 1992), which contain characteristic sequence domains (Marchler-Bauer et al., 2003), are underlined. Amino acids, which are identical in the APB24 protein and all three very long chain fatty alcohol dehydrogenases isolated from Candida species (Genbank accession numbers CAB75352, CAB75353, AAS46878), are indicated by asterisks.

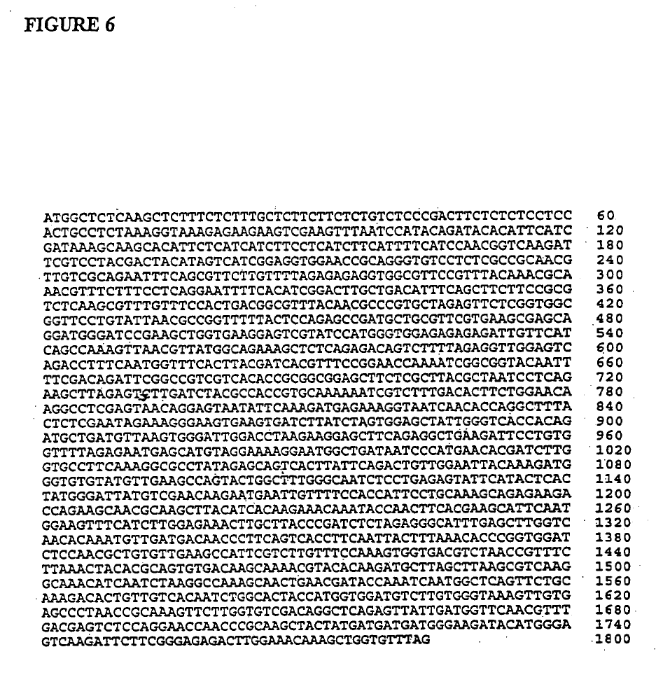

FIG. 6. (=SEQ ID NO: 1) Coding nucleic acid sequence of the APB24 gene.

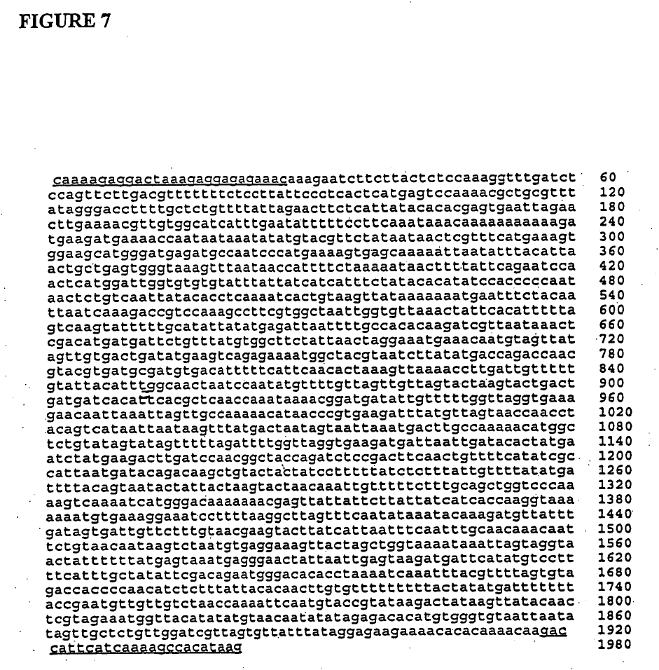

FIG. 7. (=SEQ ID NO:3) Nucleic acid sequence of the promoter of the APB24 gene.

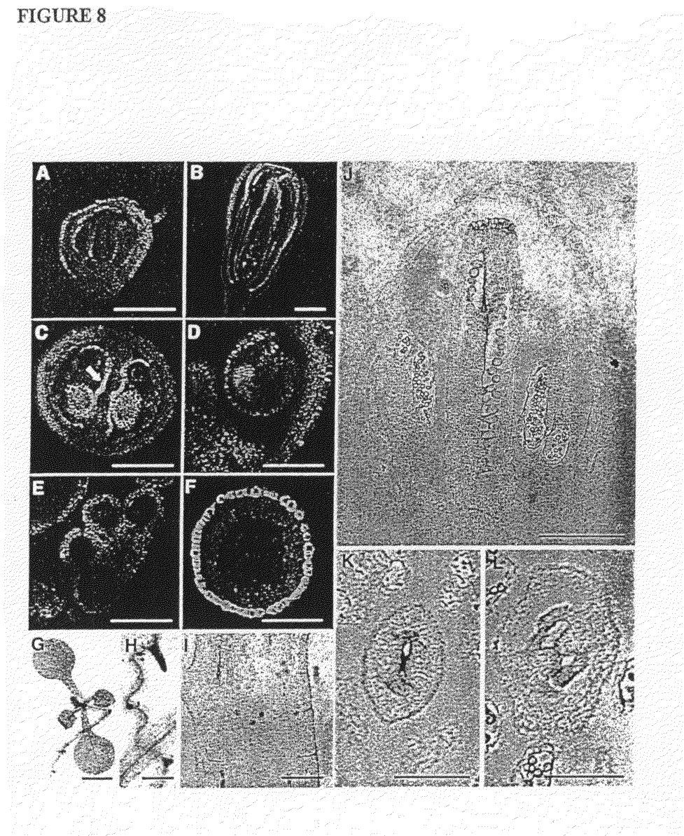

FIG. 8. Epidermis specific expression of the APB24 gene.

The term “nucleobase” as used herein refers to heterocyclic nitrogenous bases, particularly purines or pyrimidines (i.e., adenine, cytosine, thymine and guanine), which are natural constituents of nucleic acids where they occur in N-glycosidic linkage with a sugar, particularly a pentose. This term will also include heterocyclic nitrogenous bases, particularly purines or pyrimidines, which constitute unnatural peptide nucleic acids (PNAs) comprising the polyamide backbone with nucleobase side chains. Sequence specific hybridisation of nucleic acids and PNAs occurs through Watson-Crick H-bonding between the complementary nucleobases.

The term “homologue” or “homologous sequence” as used herein refers to nucleic acid or amino acid sequences with significant similarity to the APB24 sequence or parts thereof, whereby these homologous sequences show an activity comparable to the activity of the APB24 gene. In particular, as homologous sequences are considered nucleic acid sequences, of at least 21, preferably more than 50 and particularly preferably more than 200 nucleotides length, which specifically hybridise with the nucleic acid sequence of the APB24 gene or parts of this sequence under stringent or less stringent conditions (about stringent and less stringent conditions see Sambrook et al., Molecular Cloning, Cold Spring Harbour Laboratory (1989), ISBN 0-87969-309-6). An example for stringent hybridisation conditions is: hybridisation in 4×SSC at 65° C. (alternatively in 50% formamide and 4×SSC at 42° C.), followed by several washing steps in 0.1×SSC at 65° C. for one hour. An example for a less stringent hybridisation condition is hybridisation in 4×SSC at 37° C., followed by several washing steps in 1×SSC at room temperature. As homologous sequences are furthermore considered nucleic acid or amino acid sequences or parts thereof derived from different plants and showing an activity comparable to the activity of the APB24 gene, wherein said nucleic acid or amino acid sequences or parts thereof show a significant similarity with the nucleic acid and amino acid sequence of the APB24 sequence or parts thereof using the similarity algorithm BLAST (Basic Local Alignment Search Tool, Altschul et al., Journal of Molecular Biology 215, 403-410 (1990). As significant similar, as used herein, are considered sequences, which e.g. using standard parameters in Blast-Service of NCBI show a Probability of P<10⁻⁵, if they are compared with the sequence of the APB24 gene or parts thereof.

The term “specifically hybridise” and “hybridise” means that homologous nucleic acid molecules hybridise with nucleic acid molecules, which encode a protein according to the invention, or a functional fragment thereof, but not with nucleic acid molecules, which encode other proteins. In particular, the invention relates to such nucleic acid molecules, which hybridise with transcripts of nucleic acid molecules according to the invention and, as a result, can hinder their expression. Such nucleic acid molecules, which specifically hybridise with the nucleic acid molecules according to the invention, can, for example, be constituents of antisense, RNAi or co-suppression constructs or ribozymes, or can be used as primers for PCR amplification.

The term “homologue” or “homologous sequence” as used herein refers furthermore to nucleic acid or amino acid sequences or parts thereof preferably being identical to the APB24 sequence or parts thereof for at least 60%, more preferably for at least 70% or 80% or 90% and most preferably for at least 95%. The differences to the APB24 sequence or parts thereof can result from deletions, substitutions (like transitions and transversions), additions, insertions and/or inversions.

The term “identity” or “identical” as used herein refers to a sequence identity over the whole length of the coding region of nucleic acid sequences or to a sequence identity over the whole length of amino acid sequences of at least 60%, in particular an identity of at least 80%, preferably greater than 80%, particularly preferably greater than 90% and especially of at least 95%. In conjunction with the present invention, the term “identity” or “identical” is to be understood to refer to the number of amino acids/nucleotides (identity) corresponding to amino acids/nucleotides of other polypeptide sequences/nucleic acid sequences, put forward as a percentage value. Identity is preferably determined by comparing SEQ ID NO: 2 for amino acids or SEQ ID NO: 1 or SEQ ID NO: 3 for nucleic acids with other polypeptide sequences and nucleic acid sequences, respectively, with the help of computer programs. If sequences that are compared with one another have different lengths, the identity is to be determined in such a way that the amino acid and nucleotide number of the polypeptide and nucleic acid, respectively, which have the shorter sequence in common with the longer sequence, determines the percentage value. The respective formula reads as follows: (number of the amino acids/nucleotides of the longer sequence)/(number of the amino acids/nucleotides of the shorter sequence in common with the longer sequence)×100=percent by identity.

Preferably, identity is determined by means of the computer program ClustalW, which is well known and available to the public (Thompson et al., 1994). ClustalW is made publicly available by Julie Thompson (Thompson@EMBL-Heidelberg. DE) and Toby Gibson (Gibson@EMBL-Heidelberg. DE), European Molecular Biology Laboratory, Meyerhofstrasse 1, D 69117 Heidelberg, Germany. ClustalW can also be downloaded from different internet sites, including the IGBMC (Institut de Gemetique et de Biologie Moleculaire et Cellulaire, B.P.163, 67404 IIIkirch Cedex, France; ftp:/lftp-igbmc.u-strasbgJr/pub/) and the EBI (ftp://ftp.ebLac.ukipub/software/) as well as from all mirrored internet sites of the EBI (European Bioinformatics Institute, Wellcome Trust Genome Campus, Hinxton, Cambridge CB10 1SD, UK).

Preferably, Version 1.8 of the ClustalW computer program is used to determine the identity between proteins according to the invention and other proteins. In doing so, the following parameters must be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, Version 1.8 of the ClustalW computer program is used to determine the identity between the nucleotide sequence of the nucleic acid molecules according to the invention, for example, and the nucleotide sequence of other nucleic acid molecules. In doing so, the following parameters must be set: KTUPLE=2, TOPDIAGS=4, PAIRGAP=5, DNAMATRIX:IUB, GAPOPEN=10, GAPEXT=5, MAXDIV=40, TRANSITIONS: unweighted.

Furthermore, “identity” means that functional and/or structural equivalence exists between the nucleic acid molecules concerned or the proteins encoded by them. The nucleic acid molecules, which are homologous to the molecules described above and constitute derivatives of these molecules, are generally variations of these molecules, which constitute modifications, which execute the same biological function. At the same time, the variations can occur naturally, for example they can be sequences from other plant species, or they can be mutations, wherein these mutations may have occurred in a natural manner or have been introduced by objective mutagenesis. The variations can also be synthetically manufactured sequences. The allelic variants can be both naturally occurring variants and also synthetically manufactured variants or variants produced by recombinant DNA techniques. Nucleic acid molecules, which deviate from nucleic acid molecules according to the invention due to degeneration of the genetic code, constitute a special form of derivatives.

The term “corresponding” as used herein means that, in the comparison of several objects, the objects compared with one another have been kept under the same conditions. Within the scope of the present invention, the term “corresponding” in conjunction with wild type plant cells or wild type plants means that the plant cells or plants, which are compared with one another, have been raised under the same cultivation conditions and that they have the same (cultivation) age.

The term “probe” as used herein refers to nucleic acid sequences or polymer molecules containing nucleobases capable of hybridising to homologous nucleic acid sequences. The said nucleic acid sequences or polymer molecules containing nucleobases should be able to hybridise in a sequence-specific manner to the APB24 sequence or a sequence that is homologous to the APB24 sequence.

The term “derivative” as used herein refers to nucleic acid sequences encoding the APB24 protein or parts thereof, although the nucleic acid sequence differs from the naturally occurring APB24 sequence. Thus, the term derivative refers as well to genomic equivalents carrying introns as to RNA, expressed sequence tags (ESTs), cDNA sequences or to equivalents to the APB24 sequence evolved from the degeneration of the genetic code.

The term “fragment” as used herein refers to the complete amino acid sequence of the APB24 gene, or, preferably, to its fragment from amino acid position 63 to amino acid position 580 as specified in FIG. 5.

The term “vector” as used herein refers to naturally occurring or artificially created constructs for incorporation, propagation, expression or transfer of nucleic acids, e.g. plasmids, phagemids, cosmids, artificial chromosomes, bacteriophages, viruses, retroviruses.

The term “expression system” as used herein refers to any combination of vectors, restriction enzymes, transformation methods, cell extracts, living cells e.g. prokaryotic or eukaryotic cells or organisms for the purpose of endogenous or exogenous expression of genes.

The term “assay” as used herein refers to a method for the determination of the amount of a particular constituent of a mixture that contains a polypeptide encoded by the nucleic acid sequence according to SEQ ID NO:1 or homologous sequences thereof. Thus, the term includes methods for the determination of the biochemical activity of a polypeptide encoded by the nucleic acid sequence according to SEQ ID NO: 1 or homologous sequences thereof.

The term “host cells” as used herein refers to bacterial or eukaryotic cells, which do not naturally contain the nucleic acids or polypeptides encoded by the nucleic acid sequences according to SEQ ID NO:1 or homologous sequences thereof.

The term “wild type plant cell(s)” as used herein means that the plant cell(s) concerned was/were used as starting material for the manufacture of the plant cell(s) according to the invention, i.e. their genetic information, apart from the introduced genetic modification, corresponds to that of (a) plant cell(s) according to the invention.

The term “wild type plant(s)” as used herein means that the plant(s) concerned was/were used as starting material for the manufacture of the plant(s) according to the invention, i.e. their genetic information, apart from the introduced genetic modification, corresponds to that of (a) plant(s) according to the invention.

The term “transgenic plants” as used herein refers to plants produced by recombinant gene technology and/or microbiological methods but not by usual breeding methods. It is meant that cells of transgenic plants are capable of producing nucleotide or polypeptide molecules that were not produced by corresponding cells of non-transgenic plants prior to transformation. The term “transgenic plants” includes plants obtained through regeneration, vegetative and sexual reproduction.

In the present embodiment, transgenic plants may be produced from any monocotyledonous or dicotyledonous species plants, including, but not limited to, rapeseed, canola, tomato, potato, safflower, sunflower, soybean, peanut, cotton, palm, maize, sorghum, rice, barley and wheat.

The term “transgenic” as used herein refers to the introduction of homologous and/or heterologous foreign nucleic acid molecules into the genome of a plant cell or into the genome of a plant. The plant cells according to the invention or plants according to the invention are modified with regard to their genetic information by the introduction of a foreign nucleic acid molecule. The presence or the expression of the foreign nucleic acid molecule leads to a phenotypic change.

The term “phenotypic change” as used herein refers to any change detected by the comparison of transgenic and wild type plant cells or plants, the change being detectable on molecular, cellular and/or multicellular (i.e. organismic) level.

Amongst other things, transgenic plant cells and transgenic plants of the invention, can be differentiated from wild type plant cells and wild type plants, respectively, in that they contain a foreign nucleic acid molecule, which does not occur naturally in wild type plant cells or wild type plants, or in that such a molecule is present integrated at a place in the genome of the plant cell according to the invention or in the genome of the plant according to the invention at which it does not occur in wild type plant cells or wild type plants, i.e. in a different genomic environment. Furthermore, plant cells according to the invention and plants according to the invention of this type differ from wild type plant cells and wild type plants, respectively, in that they contain at least one copy of the foreign nucleic acid molecule stably integrated within their genome, possibly in addition to naturally occurring copies of such a molecule/nucleic acid sequence in the wild type plant cells or wild type plants. If the foreign nucleic acid molecule(s) introduced into the plant cells according to the invention or into the plants according to the invention is (are) additional (copy) copies of (a) molecule(s)/nucleic acid sequence(s) already occurring naturally in the wild type plant cells or wild type plants respectively, then the plant cell(s) according to the invention and the plant(s) according to the invention can be differentiated from wild type plant cell(s) or wild type plant(s), respectively, in particular in that this additional copy or these additional copies is (are) localised at places in the genome at which it does not occur (or they do not occur) in wild type plant cell(s) or wild type plant(s). This can be verified, for example, with the help of a Southern blot analysis.

Furthermore, the plant cells according to the invention and the plants according to the invention can preferably be differentiated from wild type plant cells or wild type plants, respectively, by at least one of the following characteristics: If the foreign nucleic acid molecule that has been introduced is heterologous with respect to the plant cell or plant, then the plant cell(s) according to the invention or plant(s) according to the invention have (has) transcripts of the introduced nucleic acid molecules. These can be verified, for example, by Northern blot analysis or by RT-PCR (Reverse Transcription Polymerase Chain Reaction). Plant cells according to the invention and plants according to the invention, which express an antisense and/or an RNAi transcript, can be verified, for example, with the help of specific nucleic acid probes, which are complementary to the RNA (occurring naturally in the plant cell), which is coding for the protein. Preferably, the plant cell(s) according to the invention and the plant(s) according to the invention contain a protein, which is encoded by an introduced nucleic acid molecule. This can be demonstrated by immunological methods, in particular by a Western blot analysis.

If the foreign nucleic acid molecule that has been introduced is homologous with respect to the plant cell or plant, the plant cell(s) according to the invention or plant(s) according to the invention can be differentiated from wild type plant cells or wild type plants, respectively, due to the additional expression of the introduced foreign nucleic acid molecule, for example. The plant cell(s) according to the invention and the plant(s) according to the invention preferably contain transcripts of the foreign nucleic acid molecule(s). This can be demonstrated by Northern blot analysis, for example, or with the help of so-called quantitative PCR.

The term “transformation” as used herein refers to a process of changing host DNA by transferring foreign DNA into the genome of the host. It includes the transfer of foreign DNA into the host cell followed by integration of said foreign DNA into the DNA of the host. It includes the transfer of foreign DNA into organelles of the host cell, e.g. mitochondria, plastids, or into its nucleus. The transformation as used herein can be achieved by using naked foreign DNA or in combination with other molecules that help breaking, for example, through the cell wall and membranes, transferring foreign DNA in the cell and integrating it into the host DNA.

The term “regeneration” as used herein refers to the development of whole plants from particular cells, tissues or organs. It includes also a classical definition, which means the development of whole plants from undifferentiated cells in vitro in tissue culture using solid or liquid medium supplemented will all nutrient and hormonal components necessary for plant growth.

The term “target nucleic acid” as used herein refers to a nucleic acid sequence according to SEQ ID NO:1 or homologous sequences thereof and encompasses DNA, RNA, including pre-mRNA and mRNA, transcribed from such DNA, and also cDNA derived from such RNA.

The terms “overexpression” and “overexpressing” as used herein refer to a process of obtaining transgenic plants capable of producing RNA or a polypeptide of interest either in the whole plant or in particular organs or tissues of transgenic plants. It is meant that RNA fragments may be produced in the direct (5′ to 3′) or in the opposite (3′ to 5′) orientation.

The term “expression” as used herein refers to both transcription and translation. The nucleic acid molecules according to the invention can have an in “sense” orientation and/or an “antisense” orientation with respect to the regulatory sequences.

The term “recombinant nucleic acid molecule” as used herein is to be understood to mean a nucleic acid molecule, which contains additional sequences in addition to nucleic acid molecules according to the invention, which do not naturally occur in the combination in which they occur in recombinant nucleic acids according to the invention. Here, the above mentioned additional sequences can be any sequences, preferably they are regulatory sequences (promoters, termination signals, enhancers), particularly preferably they are regulatory sequences that are active in vegetable tissue, and especially particularly preferably they are regulatory sequences that are active in vegetable tissue, in which storage starch is synthesised. Methods for the creation of recombinant nucleic acid molecules according to the invention are known to the person skilled in the art, and include genetic methods such as bonding nucleic acid molecules by way of ligation, genetic recombination, or new synthesis of nucleic acid molecules, for example (see e.g. Sambrok et al., 2001; Ausubel et al., 2002).

For expressing nucleic acid molecules according to the invention, these are preferably linked with regulatory DNA sequences, which guarantee transcription in plant cells. In particular, said regulatory DNA sequences include promoters. In general, any promoter that is active in plant cells is eligible for expression.

The term “promoter” as used herein refers to a DNA sequence containing binding sites for proteins involved in the initiation and regulation of transcription from the DNA sequence of interest. The promoter, preferably, is a natural DNA sequence upstream the sequence which encodes a protein. The term “promoter” also refers in this embodiment to synthetic sequences, which are not derived from any organism, that contain binding sites for proteins required for initiation and regulation of transcription. The specific activity of the promoter in transgenic plants, as used herein, refers to a combination of the particular promoter sequences and particular host cells, which are able to provide required proteins. It is meant that promoters can be active in all cells (e.g. ectopic promoters) or in specific cells or tissues (e.g. epidermis or seed specific promoters). It is meant also that promoters can be specifically active only in cells exposed to particular environmental conditions (e.g. water deficit) when suitable transcriptional regulation proteins become available in the cell in response to these conditions.

The promoter can be chosen so that expression takes place constitutively or only in a certain tissue, at a certain stage of the plant development or at a time determined by external influences (inducible/regulable promoter). The promoter can be homologous or heterologous both with respect to the plant and with respect to the nucleic acid molecule.

Suitable promoters are, for example, the promoter of the 35S RNA of the cauliflower mosaic virus and the ubiquitin promoter from maize for constitutive expression, the patatin promoter B33 (Rocha-Sosa et al., 1989) for tuber-specific expression in potatoes or a promoter, which ensures expression only in photosynthetically active tissues, e.g. the ST-LS1 promoter (Stockhaus et al., 1987; Stockhaus et al., 1989) or, for endosperm-specific expression of the HMG promoter from wheat, the USP promoter, the phaseolin promoter, promoters of zein genes from maize (Pedersen et al., 1982; Quatroccio et al., 1990), glutelin promoter (Leisy et al., 1990; Zheng et al., 1993; Yoshihara et al., 1996) or shrunken-1 promoter (Nerr et al., 1985).

However, promoters can also be used, which are only activated at a time determined by external influences (see for example WO 93/07279). Promoters of heat-shock proteins, which allow simple induction, can be of particular interest here. Furthermore, seed-specific promoters can be used, such as the USP promoter from Vicia faba, which guarantees seed-specific expression in Vicia faba and other plants (Fiedler et al., 1993; Baumlein et al., 1991).

Furthermore, a termination sequence (polyadenylation signal) can be present within the expression construct, which is used for adding a poly-A tail to the transcript. A function in the stabilisation of the transcripts is ascribed to the poly-A tail. Elements of this type are described in the literature (cf. Gielen et al., 1989) and can be exchanged at will.

Intron sequences can also be present between the promoter and the coding region. Such intron sequences can lead to stability of expression and to increased expression in plants (Callis et al., 1987; Luehrsen, and Walbot, 1991; Rethmeier, et al., 1997; Rose and Beliakoff, 2000; Vasil et al., 1989; XU et al., 2003,). Suitable intron sequences are, for example, the first intron of the shl gene from maize, the first intron of the polyubiquitin gene 1 from maize, thefirst intron of the EPSPS gene from rice or one of the two first introns of the PATI gene from Arabidopsis.

The terms “extracellular matrix polymers” or “ECM polymers” as used herein refer to polysaccharides, polypeptide, aliphatic and aromatic lipid molecules that are cross-linked in a network outside the cellular membrane of the plant cell. The above mentioned terms shall comprise insoluble materials of cell wall, cuticle, suberin, lignin and sporopollenin.

The term “substrate”, as used herein, refers to a chemical substance that can be modified due to enzymatic activity of the polypeptide encoded by the nucleic acid sequence according to SEQ ID NO: 1 or a homologous nucleic acid sequence thereof. It refers but is not limited to ω-hydroxyfatty acids having 8 to 40 carbon atoms that are natural substrates of the polypeptides described herein. Preferably, it refers to ω-hydroxyfatty acids having 14 to 30 carbon atoms, and more preferred to ω-hydroxyfatty acids having 14 to 24 carbon atoms, further to ω-hydroxyfatty acids having 16 to 24 carbon atoms, and further to ω-hydroxyfatty acids having 14 to 20 carbon atoms.

The term “product”, as used herein, refers to a chemical substance that can be produced from a substrate due to enzymatic activity of the polypeptide encoded by the nucleic acid sequence according to SEQ ID NO:1 or a homologous nucleic acid sequence thereof. Preferably, it refers but is not limited to ω-oxofatty acids having 8 to 40 carbon atoms that are natural products of the polypeptides described herein. Preferably, it refers to ω-oxofatty acids having 14 to 30 carbon atoms, and more preferred to ω-oxofatty acids having 14 to 24 carbon atoms, further to ω-oxofatty acids having 16 to 24 carbon atoms, and further to ω-oxofatty acids having 14 to 20 carbon atoms.

The term “inhibitor”, as used herein, refers to a chemical substance that inactivates the enzymatic activity of the polypeptides encoded by the nucleic acid sequence according to SEQ ID NO:1 or a homologous nucleic acid sequence thereof. The inhibitor may function by interacting with a cofactor of the enzyme, the substrate of the enzyme, or preferably by interacting directly with the enzyme.

The terms “biological activity” or “enzymatic activity”, as used herein, refer to the natural enzymatic activity of the polypeptides encoded by the nucleic acid sequence according to SEQ ID NO:1 or a homologous nucleic acid sequence thereof, wherein said enzymatic activity may be directly or indirectly measured by methods known by the skilled artisan, and wherein the measurement allows to estimate the catalytic activity of said enzymatically active polypeptides.

The terms “herbicide” or “herbicidal compound”, as used herein, refer to inhibitors as defined above, and which may be used to kill or suppress the growth of plant cells, tissues or a whole plant.

The present invention relates to the APB24 gene which encodes a very long chain fatty alcohol dehydrogenase involved in the biosynthesis of α-,ω-dicarboxylic fatty acids naturally required for the proper formation of the cuticular layer of the cell wall in plants and the cuticle.

Based on this finding, the present invention refers to methods for producing plants with modified properties, comprising stable integration of at least of the nucleic acid sequence according to SEQ ID NO:1, or a homologous nucleic acid sequence thereof, or of a fragment or derivative thereof, having the biological activity of a polypeptide encoded by the nucleic acid sequence according to SEQ ID NO:1, into the genome of plant cells or plant tissues and regeneration of the obtained plant cells or plant tissues to plants.

Conventional technologies for producing stable integration of foreign nucleic acid sequences into nucleic acid sequences of host cells are based on electroporation, direct DNA uptake, infectious agents, microinjection, and particle bombardment as described in a number of manuals: Plant Genetic Transformation and Gene Expression: A Laboratory Manual, J. Draper, R. Scott, P. Armitage, R. Walden, 1988, Blackwell Pub; Plant Cell Electroporation and Electrofusion Protocols (Methods in Molecular Biology, 55, J. A. Nickoloff, 1995, Humana Press; Gene Transfer to Plants (Springer Lab Manual), I. Potrykus, G. Spangenberg, 1995, Springer-Verlag Telos; Particle Bombardment for Genetic Engineering of Plants (Biotechnology Intelligence Unit), P. Christou, 1996, R. G. Landes; Genetic Transformation of Plants (Molecular Methods of Plant Analysis New Series), J. F. Jackson, H. F. Linskens, 2003, Springer-Verlag.

The choice of the particular method to be used to transform host cells depends on the species of plant, and these methods are not critical to the current invention. Any appropriate method or combination of methods resulting in the stable integration and expression of the sequence or sequences described in this invention in the desired host cells is acceptable. For example, as known to a person skilled in the relevant art, the use of Agrobacterium tumefaciens-mediated co-transformation using T-DNA binary vectors is the method of choice for many dicotyledonous plants, because it appears to result in high transformation efficiencies and in more predictable segregation patterns of the transgenes when compared to the electroporation or particle delivery systems. With this method single or multiple insertions of the transgene can be obtained that are randomly distributed throughout the nuclear genome.

One aspect of the present invention is a transgenic plant with modified extracellular matrix polymers, e.g. cuticle, having changed diffusivity or strength, thereby displaying an enhanced resistance against e.g. air pollution and pesticides. Said changed diffusivity or strength may result from both increase of the amount of the APB24 protein or RNA and from reduction of the level of the APB24 protein or RNA. Changed diffusivity of the cuticle may be achieved by overexpressing the polypeptide encoded by the nucleic acid sequence according to SEQ ID NO:1 or a homologous nucleic acid sequence thereof in the epidermis of transgenic plants.

Changed diffusivity of extracellular matrix polymers may be regarded as the capacity of preventing water loss through the surface of the plant or as the capacity of reducing speed of chemical diffusion across modified extracellular matrix polymers, e.g. the cuticle. Diffusive properties of the cuticle may be quantified using previously published methods, which estimate the transpiration of water, gas exchange or penetration of radioactively labelled molecules through the cuticle (Niederl et al., 1998; Santrucek et al., 2004; Vogg et al., 2004).

Changed strength of extracellular matrix polymers, e.g. the cuticle, may be regarded as the capacity of preventing mechanical, biological or chemical damage of the plant as a whole or fruits and vegetables. In these particular cases, extracellular matrix polymers, e.g. cuticles and epidermal cell walls of transgenic plants obtained according to the present invention, may have more or less α-,ω-dicarboxylic fatty acids that would improve cross-linking in the cell wall and in cuticular polymers. Improved cross-linking should be beneficial to plants by enhancing protective properties of extracellular matrix polymers, e.g. the cuticle and the outermost cell wall of transgenic plants.

Correspondingly, transgenic plants obtained according to the present invention, may have enhanced drought or disease resistance, improved shelf life and transportation behaviour of fruits and vegetables. They also may have reduced scent of flowers, leaves, fruits or roots of the plant. Conversely, reduced cross-linking should result in lessening protective properties of extracellular matrix polymers, e.g. the cuticle and the outermost cell wall of transgenic plants.

Correspondingly, in yet another embodiment, changed strength of the cuticle may be regarded as the capacity of emitting more odorous substances that enhances scent of flowers, leaves, fruits or roots of the transgenic plant. This can be achieved for example by overexpressing the nucleic acid sequence according to SEQ ID NO:1 or a homologous nucleic acid sequence thereof in the epidermis of transgenic plants in the direct (5′ to 3′) or, preferably, in the opposite (3′ to 5′) orientation, or, most preferably, in both.

This approach is based on experimental data, known to a person skilled in the art, that the overexpressing nucleic acid may interfere with normal functions of the target nucleic acid through specific hybridisation and result in the phenomena known as co-suppression, transcriptional and posttranscriptional silencing and RNA interference (Cogoni and Macino, 2000; Hammond et al., 2001; Schramke and Allshire, 2004). The normal functions of DNA to be interfered with may include replication and transcription. The normal functions of RNA to be interfered with may include splicing of the RNA, translocation of the RNA to the site of protein synthesis and translation of protein from the RNA. As a result of such interference, transgenic plants overexpressing the nucleic acid sequence according to SEQ ID NO:1 or a homologous nucleic acid sequence thereof may not produce or may not produce the sufficient amount of the corresponding polypeptide and, accordingly, may develop the cuticle incapable of reducing the emission of odorous substances that will enhance scent of flowers, leaves, fruits or roots of the transgenic plant.

Another aspect of the present invention is a genetically modified plant having a changed lipid composition.

This may be achieved by overexpressing the polypeptide encoded by the nucleic acid sequence according to SEQ ID NO:1 or a homologous nucleic acid sequence thereof in the plant organs which are able to accumulate vegetable oil (triacylglycerols) or waxes (esters of very-long-chain (greater than 18 carbons) mono-unsaturated fatty acids) and fatty alcohols, for storage (e.g. seeds). Seeds may be preferably used as they are able to accumulate relatively high amount of oil that can be easily extracted and concentrated, and thus produced at a very low cost.

For this application, native or artificial promoters may be used that allow to express the transgene in the organ or tissue of interest (reviewed by Potenza et al., 2004). For example, seed specific expression can be achieved under control of the napin promoter from Brassica napus (Broun and Somerville, 1997) or Arabidopsis thaliana (Smith et al., 2000) and the hydroxylase promoter from Lesquerella fendleri (Broun et al., 1998).

To make use of the very long chain fatty acid ω-alcohol dehydrogenase, which is the subject of this invention, the cells or tissues, in which the said APB24 gene will be overexpressed in transgenic plants, must contain ω-hydroxylated fatty acids that are natural substrates of the enzyme. While these molecules are typically synthesised by the cells producing extracellular matrix polymers, they do not accumulate in the seed oil (the database “Seed oil fatty acids” (SOFA), http://www.bagkf.de/html/sofa.html). Various approaches could be used for engineering of transgenic plants capable of producing ω-hydroxylated fatty acids in the seed oil, and they are not a subject of this invention. One approach could be to express in seeds fatty acid cytochrome P450s monooxygenases, capable of oxidising ω-carbon of fatty acids, such as, for example, fatty acid ω-hydroxylases of the CYP86 and CYP94 gene families (Benveniste et al., 1998; Le Bouquin et al., 1999; Pinot et al., 1999; Wellesen, 2001). Examples that show how seed oil composition can be changed to contain hydroxylated fatty acids have been given (Broun and Somerville, 1997; Smith et al., 2000). Also, the use of plant fatty acyl hydroxylases to produce hydroxylated fatty acids and derivatives in plants is a subject of patent applications U.S. Pat. No. 6,028,248 and U.S. Pat. No. 6,433,250.

As a part of the present invention, ω-hydroxylated fatty acids may be specifically oxidised at the ω-carbon through the reaction catalysed by the invented polypeptide in planta. Products of this reaction, fatty acids bearing ω-aldehyde, either in a free form or bound, may be extracted from plants, e.g. seeds of the transgenic plants, and refined. The extraction and refinement, e.g. of oil or wax, can be achieved by heat, solvents, or pressure as described by Gunstone and Hamilton (2001).

Also, products of the reaction, catalysed by the invented very long chain fatty ω-alcohol dehydrogenase, may be further oxidised in planta to more stable α,ω-dicarboxylic fatty acids by enzymes present in the cells. In principle, such enzymes can be endogenous to plant cells or can be made to be expressed in appropriate host cells or tissues of the plant. For example, enzymes capable of oxidising fatty acids, which bear ω-aldehyde, are CYP94A5, a cytochrome P450 from Nicotiana tabacum (Le Bouquin et al., 2001) and the aldehyde dehydrogenase from rabbits (Ichihara et al., 1986a; Ichihara et al., 1986b). The products, α,ω-dicarboxylic fatty acids, can be obtained in the plant either in a free form or bound, and could be a part of e.g. oil or wax. Their extraction and refinement are not subjects of the present invention and can be achieved as described by Gunstone and Hamilton (2001).

It is essential in this embodiment that fatty acids obtained from genetically modified plants, tissues or cells get a reactive group, preferably aldehyde or carboxyl group, at the omega side of the aliphatic chain due to activity of the very long chain fatty ω-alcohol dehydrogenase described by this invention. Having reactive groups at both sides of the aliphatic chain adds bifunctionality and allows to use such molecule in cross-linking reactions, in particular, for production of polyesters and monoesters.

The types of reactions and methods of cross-linking are not critical to the current invention. The following patents exemplify the use of aliphatic α,ω-dicarboxylic acids for production of polyesters and monoesters, nylons, fibers, including biodegradable materials, and for the preparation of intermediates for medicines and agricultural chemicals, perfumes, lubricants and adhesives: U.S. Pat. No. 5,962,285; U.S. Pat. No. 6,750,286; U.S. Pat. No. 6,639,045; U.S. Pat. No. 6,417,266; U.S. Pat. No. 6,358,620; U.S. Pat. No. 6,268,465; U.S. Pat. No. 6,350,850; U.S. Pat. No. 6,200,640; U.S. Pat. No. 6,211,317; U.S. Pat. No. 6,355,830; U.S. Pat. No. 6,485,819; U.S. Pat. No. 6,149,995; U.S. Pat. No. 6,750,318; U.S. Pat. No. 6,127,080; U.S. Pat. No. 6,479,618; U.S. Pat. No. 6,468,319; U.S. Pat. No. 6,071,599; U.S. Pat. No. 5,780,582; U.S. Pat. No. 5,962,624; U.S. Pat. No. 6,562,939; U.S. Pat. No. 6,458,858; U.S. Pat. No. 6,120,895; U.S. Pat. No. 5,955,529.

Currently, chemical synthesis of α,ω-dicarboxylic fatty acids is the method of choice, and several chemical routes are available as exemplified by the following patents: U.S. Pat. No. 6,437,180; U.S. Pat. No. 5,973,173; U.S. Pat. No. 5,696,303.

However, most chemical methods result in mixtures of molecules, and extensive purification steps are required, especially, to prepare long-chain α,ω-dicarboxylic fatty acids. Therefore, production of dicarboxylic acids using special microorganisms can be advantageous. The following patents disclose the use of the microbiological approach: U.S. Pat. No. 4,474,882; U.S. Pat. No. 3,975,234; U.S. Pat. No. 4,965,201; U.S. Pat. No. 4,339,536; U.S. Pat. No. 5,962,285; U.S. Pat. No. 5,620,878; GB 1 405 026; DE 2 164 626; DE 2 853 847; DE 2 937 292; DE 2 951 177; DE 2 140 133.

Overexpressing the polypeptide encoded by the nucleic acid sequence according to SEQ ID NO:1 or a homologous nucleic acid sequence thereof in the transgenic plant will provide another way to produce modified fatty acids that carry a reactive group, preferably aldehyde or carboxyl group, at the omega side of the aliphatic chain, based on renewable resources, e.g. seed oil.

Another aspect of the present invention includes the in vitro or in vivo production of the polypeptide encoded by the nucleic acid sequence according to SEQ ID NO:1 or a homologous nucleic acid sequence thereof.

In vivo production can include the use of host cells expressing the nucleic acid sequence according to SEQ ID NO:1 or a homologous nucleic acid sequence thereof. Various vectors for cloning and expression of a polypeptide in a variety of different host cells currently available (for further details see, for example, Molecular Cloning: a Laboratory Manual: 3rd edition, Sambrook et al., 2001, Cold Spring Harbor Laboratory Press) and they are not critical to the current invention.

In vitro production can include the use of in vitro translation or transcription/translation systems such as in vitro Express™ Translation Kit (Stratagene), TnT Quick Coupled Transcription/Translation System (Promega), Rapid Translation Systems (RTS) from or alike cell-free protein expression systems that comprise an extract prepared from the intracellular contents of cells. These extracts generally contain those molecules which support transcription and protein translation and can be prepared for e.g. from Escherichia coli cells, wheat germ cells, rabbit reticulocytes, insect cells and frog oocytes by a person skilled in the art according to manuals (e.g. In Vitro Transcription and Translation: 1st edition, M. J. Tymms, 1995, Humana Press).

Another aspect of the present invention includes the use of the polypeptides encoded by the nucleic acid sequence according to SEQ ID NO:1 or a homologous nucleic acid sequence thereof for enzymatic reactions with appropriate substrates or in assays. This includes using the polypeptides encoded by the nucleic acid sequence according to SEQ ID NO:1 or a homologous nucleic acid sequence thereof in screening assays to identify inhibitors.

The ω-hydroxyfatty acid dehydrogenase encoded by the nucleic acid sequence according to SEQ ID NO:1 or a homologous nucleic acid sequence thereof converts long-chain ω-hydroxy fatty acids, which are natural substrates in planta, to fatty aldehydes. For enzymatic reactions, the said polypeptide expressed in host cells or cell-free protein expression systems can be incubated in a reaction buffer with a chemical compound which is not necessarily a natural substrate. For example, any organic compound carrying a hydroxyl group that can be oxidised to aldehyde group due to enzymatic activity of the polypeptides described herein can be used for enzymatic reactions in vitro or in assays. Organic compounds may be characterised by the presence of carbon-hydrogen bonds, saturated or unsaturated chains of carbon, aromatic hydrocarbons and various functional groups. Millions of such compounds are known that constitute the study of organic chemistry (for further details see, for example, Organic Chemistry: 5th Edition, L. G. Wade, L. G., Jr. Wade, 2002, Prentice Hall) and they are not critical to the current invention.

It is a part of this embodiment, that enzymatic reactions or assays with the polypeptides encoded by the nucleic acid sequence according to SEQ ID NO:1 or a homologous nucleic acid sequence thereof, or cells expressing the said polypeptides are used for testing which chemical compounds are capable of acting as enzyme substrates.

For enzymatic reactions in vitro, the reaction buffer may include various components such as buffering components, salts, cofactors (e.g. nicotinamide adenine dinucleotide (NAD)), stabilising agents (e.g. inert proteins), microsomal membranes, which are added to keep the enzyme in the active state, to facilitate enzymatic reactions. Various compositions of the reaction buffer are possible, and this is not critical to the current invention. An example of the reaction buffer for plant ω-hydroxyfatty acid dehydrogenase has been provided (Agrawal and Kolattukudy, 1978a; Agrawal and Kolattukudy, 1978b).

It is a part of this embodiment, that host cells expressing the polypeptides encoded by the nucleic acid sequence according to SEQ ID NO:1 or a homologous nucleic acid sequence thereof can be used for testing which chemical compounds are capable of acting as enzyme substrates.

In addition, the above assay may include various chemical inhibitors, preferably organic compounds. It is a part of this embodiment, that enzymatic reactions or assays with the polypeptides encoded by the nucleic acid sequence according to SEQ ID NO:1 or a homologous nucleic acid sequence thereof are used for testing which chemical compounds are capable of acting as enzyme inhibitors.

It is a part of this embodiment, that host cells expressing the polypeptides encoded by the nucleic acid sequence according to SEQ ID NO:1 or a homologous nucleic acid sequence thereof can be used for testing which chemical compounds are capable of acting as enzyme inhibitors.

The course of enzymatic reactions in vitro or in the host cells can be followed using various approaches, direct and indirect methods and technologies that are not critical to the current invention (for further details see, for example, High Throughput Screening: Methods and Protocols (Methods in Molecular Biology, 190), W. P. Janzen, 2002, Humana Press; Integrated Drug Discovery Technologies, Houng-Yau Mei, A. W. Czarnik, 2002, Marcel Dekker; High Throughput Screening: The Discovery of Bioactive Substances, J. P. Devlin, 1997, Marcel Dekker).

For example, as it is known to a skilled person in the art, in order to identify inhibitors it is possible to incubate an enzymatic reaction mix or a cellular constituent, e.g. microsomal membrane, or host cells containing the polypeptides to be used according to the invention, together with a labelled substrate in the presence and absence of a candidate chemical compound, which may be capable of acting as enzyme inhibitor. The inhibitory ability of the candidate chemical compound may evident from an increased or reduced conversion of the labelled substrate.

In the other example, the activity of the enzyme in the presence and absence of a candidate chemical compound, which may be capable of acting as enzyme inhibitor, can be determined by a spectrophotometric assay for conversion of the cofactor present in the enzymatic reaction. In particular, as it is known to a skilled person in the art, the increase in absorbance at 340 nm, caused by the reduction of NADP⁺ to NADPH is a measure of the catalytic activity of many dehydrogenases.

In the other example, the activity of the enzyme in the presence and absence of a candidate chemical compound, which may be capable of acting as enzyme inhibitor, can be evident from the amount of product accumulated in the enzymatic reaction. In particular, as it is known to a skilled person in the art, ω-oxofatty acids, which are the natural products of enzymatic activity of the polypeptides to be used according to the invention, can be derivatised with 2,4-dinitrophenyl-hydrazine (DNPH), which forms derivatives with aldehydes and ketones. The increase in absorbance at 360-365 nm provides a measure of the formation of oxofatty acids (for further experimental details see, for example Weichert H. et al., FEBS Letters 464 (1999) 133-137), and allows to estimate catalytic activity of the enzyme and inhibitory effect of a candidate chemical compound.

In the other example, candidate inhibitory compounds can be identified by the screening procedures that are based rather on protein-ligand interaction than on enzymatic assays. The binding of chemical compounds, which are relatively small molecules, to the polypeptides to be used according to the invention, can be evaluated using a number of various approaches and methods, which are known to a skilled person in the art. The detection can be based on but not limited to surface plasmon resonance (SPR) recording, fluorescence correlation spectroscopy and surface-enhanced laser desorption/ionisation analysis (for further details see, for example, Single-Molecule Detection in Solution Methods and Applications, C. Zander, J. Enderlein, R. A. Keller (Eds), 2002, Wiley-VCH; Fluorescence Correlation Spectroscopy, R. Rigler, R. Rigler, E. S. Elson, 2001, Springer-Verlag; Quantitative Analysis of Biospecific Interactions, P. Lundahl, A. Lundqvist, E. Greijer, 1998, Gordon & Breach Science Publishers).

It is a part of this embodiment, that chemical inhibitors identified as described above, may be herbicidal compounds that can be used to kill or suppress the growth of plant cells, tissues or a whole plant.

With regard to the protection of the plant body against water loss, pathogens, solar radiation, pollutants and industrial chemicals extracellular matrix polymers are absolutely essential (reviewed in Kolattukudy, 2001a; Kolattukudy, 2001b; Kolattukudy, 2001c). Therefore, the suppression of ω-hydroxyfatty acid dehydrogenase enzymatic activity of the polypeptides to be used according to the invention, can result in the cell death or the death of the plant, or suppression of growth and development. With this respect, the said polypeptides can be potential herbicide targets.

Therefore, another aspect of the present invention includes the use of the polypeptides encoded by the nucleic acid sequence according to SEQ ID NO:1 or a homologous nucleic acid sequence thereof in the screening for chemical inhibitors as described above when these chemical inhibitors are to be used as herbicides. Once an inhibitor has been identified, the next step can be to apply it to plants at various concentrations. After application of the inhibitor, preferably by spraying, its herbicidal effects on plants can be recorded, including cell death, and suppression of growth and development.

Another aspect of the present invention resides in the use of an inhibitory acting compound preferably identified by a method as described above for controlling undesirable vegetation. Such use comprises the application of at least one compound having an inhibitory effect on fatty acid ω-alcohol dehydrogenase to said undesirable plants and/or their seeds. The inhibitor may be applied not only to said plants or seeds, but also to the areas where said plants grow.

### EXAMPLE 1

### Cuticular Defects Associated with the Loss of the APB24 Function (FIG. 1)

The transposon-induced mutant APB24 of Arabidopsis shows severe cuticular defects, upon inspection of the epidermis by transmission electron microscopy. For transmission electron microscopy, plant materials were fixed with a mixture of glutaraldehyde (2%) and formaldehyde (4%) in sodium cacodylate (0.05 M, pH 7.2) for 2 hr and postfixed with OsO₄ (1%) in sodium cacodylate at 4° C. overnight, dehydrated with ethanol and embedded in LR White resin (London Resin Company Ltd., UK) at 60° C. for 24 h. Ultrathin sections (50-100 nm) were double-stained with uranyl acetate (5%) for 30 min and Reynolds lead citrate for 2 min, and then examined with a Zeiss EM 10C microscope.

Cuticular defects associated with the loss of the APB24 function are evident by comparing FIG. 1A and FIG. 1B, 1C. The dark layer above the light cell wall (cuticle proper) is continuous in a wild type plant (FIG. 1A) and discontinuous (FIG. 1B) or multilayered (FIG. 1C) in the APB24 mutant. In the mutant, the direct contact of cell wall material is possible, therefore cell wall fusions between distinct organs occur as illustrated in the FIG. 1C.

This example shows that activity of the very long chain fatty ω-alcohol dehydrogenase described by this invention is required for the formation of the cuticle proper at the outermost cell walls of the epidermis.

### EXAMPLE 2

### Metabolic Analysis (FIG. 2)

With the use of GC-MS, fatty acid metabolites were compared in the APB24 mutants and wild type plants.

In the first experiment, lipids were isolated from floral buds as described previously (Browse et al., 1986) with modifications. Briefly, 30 mg of lyophilised tissues were incubated in closed glass tubes with 1 ml 1N methanolic-HCl (Supelco) and 5% (v/v) 2,2-dimethoxypropan. Before incubation for 1 h at 80° C., the tubes have been flashed with argon. The reaction mixture was then cooled to room temperature and extracted with shaking for 10 min with 0.3 ml heptane in the presence of 1 ml 0.9% NaCl. The water and organic phases were separated by centrifugation at 800 rpm for 5 min. The heptane phase was transferred into a GC microvial and analysed with a Hewlett Packard 5890 series II gas chromatograph coupled to the HP 5973 Mass Selective Detector. An HP-5MS methyl phenyl silicone capillary column (10 m×0.20 mm) was used.

Chromatographic parameters were: injector temperature 250° C.; initial oven temperature 150° C. for 1 min; the temperature was then raised to 320° C. at 12° C./min, and held there for 5 min. Peak areas were corrected before comparison with the area of the α-linolenic acid peak, which was used instead of an internal standard.

With this method, under conditions known to cleave ester bonds, extraction and transesterification of total fatty acids occurred in one step. Free fatty acids and fatty acids linked by ester bonds e.g. to glycerol, aliphatic and aromatic alcohols, extracellular matrix polymers, including cutin and suberin, were extracted into organic phase for analysis.

Fatty acid metabolites were identified by their retention times and their mass spectra, and the ratios of peak areas in APB24 and wild type were compared (FIG. 2A). While ratios close to 1 were obtained for most of the compounds, the ratios for five metabolites were altered by up to two-fold (FIG. 2B). To establish the molecular identities of these metabolites, their mass spectra were compared to those of reference substances in mass spectra libraries. One compound showed a marked increase in the APB24 mutant, and was identified as 18-hydroxyoctadecadienoic acid (peak 3), while co-oxo fatty acids with aliphatic chains of 16 and 18 carbons in length (peaks 2 and 5, respectively) were present in reduced amounts. The other compounds whose levels were lower in the mutant were the end products of ω-oxidation —C16 and C18 α-,ω-dicarboxylic acids (peaks 1 and 4, respectively). These results clearly indicate that the APB24 mutant is defective in the oxidation of long-chain ω-hydroxy fatty acids to ω-oxo fatty acids.

In the second experiment, the insoluble polymeric material isolated from leaves of the APB24 mutant and wild type plants was analysed.

For this analysis, leaves from wild type and mutant plants were thoroughly steeped in organic solvents to extract all soluble lipids. The remaining polyester lipids were subjected to hydrolysis and analysed by GC-MS.

Briefly, wild types and progeny of heterozygous plants were grown until flowering to identify APB24 mutants. Mature leaves (about 300 mg per sample) were harvested and their areas measured by scanning. Soluble lipids were extracted from samples by steeping in 25 ml of methanol/chloroform (1:1) mixture for 7 days, changing the solvent daily. After drying, the leaf material was weighed (typically about 30 mg) and stored or used directly for analysis. Transesterification of the residual bound lipids was performed with 6 ml of 1 N HCl in methanol (2 hr, 80° C.) in a Teflon-sealed screw-cap tube. The hydrophobic monomers were subsequently extracted into hexane (three times with 6 ml) containing 20 μg of dotriacontane (C32) as an internal standard. After evaporating the solvent to 100 μl at 50° C. under a stream of N₂, free hydroxyl groups were converted into their trimethylsilyl ethers with 20 μl of N,N-bis-trimethylsilyltrifluoroacetamide (BSTFA) and 20 μl of pyridine (40 min, 70° C.). Samples were analysed with GC-MS using a gas chromatograph 6890N (DB-1.30 m×0.32 mm, 0.1 μm (J&W) equipped with a quadropole mass selective detector 5973N (Agilent Technologies, Böblingen, Germany), Quantitative determination of lipids was carried out with the same GC system equipped with a flame ionisation detector (FID).

With this method, only residual bound lipids of extracellular matrix polymers, were analysed. After cleaving ester bonds and transesterification with methanolic HCl similar to that described above in the first experiment, free hydroxyl groups were derivatised by silylation with BSTFA to optimise separation of metabolites and enable their identification.

Many of the fatty acid derivatives liberated from non-solubilisable leaf lipids were constituents commonly found in both cutin and suberin, but di-unsaturated C18 fatty acids were also detected (FIG. 2C). No ω-oxo fatty acids were found in the non-solubilisable leaf lipids, suggesting that they are converted into α-,ω-dicarboxylic fatty acids before incorporation into polyesters. Lower than normal levels of α-,ω-dicarboxylic fatty acids, and elevated levels of ω-hydroxy fatty acids, were identified in the leaf polyesters isolated from the APB24 mutant (FIG. 2D).

This example shows that the loss of activity of the very long chain fatty ω-alcohol dehydrogenase described by this invention results in the reduction in the levels of α-,ω-dicarboxylic fatty acids in the insoluble polymeric material isolated from the APB24 mutant. Therefore, the deformation of the cuticle proper at the outermost cell walls of the epidermis must be attributed to the inability to cross-link extracellular lipids in the cell wall or/and in the cuticle proper.

In the third experiment, composition of residual bound lipids and cutin from B. oleracea were compared. The aim of this experiment was to find out whether composition of residual bound lipids, analysed as described in the second experiment, and composition of cutin are different. In Arabidopsis, isolated cutin often contains cell-wall contaminants, and it is difficult to define which α-,ω-dicarboxylic fatty acids are cell wall constituents and which are derived from cutin monomers. We therefore compared the chemical composition of cutin with that of the insoluble polymeric material in leaves of a close relative of Arabidopsis, Brassica oleracea var. italica (broccoli), in which isolation of cutin is feasible. For this analysis, plants were grown in the open, and leaf discs (18 mm diameter) were punched out with a cork borer using freshly harvested leaf material. Cuticular membranes were enzymatically isolated (based on J. Schoenherr, M. Riederer, Plant Cell Environ. 9, 459-466 (1986)) from adaxial leaf surfaces by incubating in a mixture (1:1) of cellulase (Celluclast, Novo Nordisk, Bagsvaerd, Denmark) and pectinase (Trenolin, Erbslöh, Geisenheim, Germany) at a concentration of 1% (w/w). After a few days, cuticles were collected and air-dried. To prepare polymer matrix membranes (cutin), cuticle disks were cleared in chloroform, which extracts soluble lipids e.g. cuticular waxes. Samples for analysis of residual bound lipids were obtained from leaf disks by steeping as described in the second experiment.

Polymer matrix membranes (cutin) and the insoluble polymeric material isolated from leaf disks were analysed with GC-MS as described in the second experiment of this example. This experiment revealed that saturated α-,ω-dicarboxylic fatty acids in the insoluble polymeric material are derived from cutin, while unsaturated α-,ω-dicarboxylic fatty acids are not (FIG. 2E). It follows, therefore, that the very long chain fatty ω-alcohol dehydrogenase described by this invention is involved in the biosynthesis of both cell-wall bound and cutin lipids, the composition of which is not necessarily identical to each other.

### EXAMPLE 3

### Expression in the Epidermis

The aim of these experiments was to study whether the APB24 gene specifically acts in dermal cells.

In the first experiment, mRNA in situ hybridisation was performed with a probe to the APB24 gene.

In situ hybridisation was performed mainly as previously described (Huijser et al., 1992) with modifications. Tissues were immersed in 100 mM phosphate buffer, pH 7.2 (prepared as 68.4 parts of Na₂HPO₄ plus 31.6 parts of NaH₂PO₄) containing 4% para-formaldehyde for fixation and evacuated several times for 15 min. until the tissue sinks. To remove fixative the tissue was rinsed in 3×15 min in the 100 mM phosphate buffer. After dehydration in series of ethanol (EtOH) solutions from 30 to 70% the tissue was incubated overnight or longer in 70% EtOH until complete destaining. Dehydration was completed in 85%, 95% and 100% EtOH for 1 hour each.

Clearing was performed in series of EtOH:Histoclear mixtures (2:1, 1:1, 1:2) for 30 min each time and in 100% Hystoclear for 4 hours at RT. Infiltration with paraffin (Paraplast Plus® from Oxford Labware) was conducted in an vacuum oven at 60° C. 6 changes of Hystoclear:paraffin (1:5) were made over 3 days.

Before sectioning the samples were evacuated and rapidly cooled in ice-cold water. Blocks of paraffin embedded tissue were cut to 7-8 μm sections on a microtome, placed on glass slides and dried on a slide warmer at 42° C. for 24-48 hours.

The digoxigenin-labelled RNA probe was synthesised from a 1600-nt PCR product prepared from a cDNA by PCR with the ACE-SP6 (CTCGAGTTTAGGTGACACTATAGAACTGGAGCCTCCACTGCCTCTAAAGGTAAAGAG) (SEQ ID NO:4) and ACE-T7 (CTCGAGTAATACGACTCACTATAGGGAGACACCAAGAACTTTGCGGTTAGGG) (SEQ ID NO:5) primers (the SP6 and T7 promoters are underlined) using DIG RNA Labelling Mix (Boehringer Mannheim) according to provided protocol.

Paraffin was removed from sections before hybridisation in series of 5 min. incubations in HystoClear:EtOH mixtures (3×1:0, 1×1:1, 1×0:1) in a staining slide holder. The sections were rehydrated by dipping them in EtOH solutions prepared with 0.85% NaCl (sequentially in 95%, 85%, 70%, 50% and 30% EtOH) and finally in 0.85% NaCl for 2 min each time. Denaturation with 0.2N HCl, Proteinase K digestion and dehydration were performed as described (Huijser et al., 1992).

Sections covered with hybridisation solution and coverslips were incubated overnight at 50° C. in a humid chamber. Posthybridisation washing and RNAse treatment was done as described (Huijser et al., 1992). Immunological detection was performed using anti-DIG AP conjugates (Boehringer Mannheim) according to manufacturer's instructions. Staining reaction with Nitro Blue Tetrazolium chloride (NTB) and 5-Bromo-4-chloro-3-indolyl,4-toluidine salt (BCIP) was completed after 12-14 hour incubation. Cell walls were optionally stained for 20 min with Calcofluor® (Sigma). Slides were dried and mounted for storage using Entellan (Merck). Sections were viewed under fluorescent light or under bright-field illumination with a Zeiss Axiophot microscope.

As it is evident from FIG. 8I-8L, the expression of the APB24 gene was found to be specific to the protoderm and epidermis in all organs studied.

In the second experiment, the promoter of the APB24 gene has been localised to the 5′ upstream region.

The 1.9-kb promoter of the APB24 gene was obtained by PCR with the primers ACE-Hind (AAAAAAAGCTTACAAAAGAGGACTAAAGAGGAGAGAAAC) (SEQ ID NO:6) and ACE-Xba (AAAAAATCTAGACTTATGTGGCTTTTGATGAATGGTC) (SEQ ID NO:7), which introduced HindIII and XbaI cloning sites (underlined), respectively, at the 5′ and 3′ ends of the promoter.

Two APB24 promoter-reporter binary vectors containing the β-glucuronidase (GUS) or green fluorescence protein (GFP) mgfp5-ER (Haseloff and Siemering, 1998) reporter gene, were constructed in the binary vector pBHS (Wellesen et al., 2001).

Transgenic plants of Arabidopsis were obtained as described (Bechtold et al., 1993) by vacuum infiltration with Agrobacterium tumefaciens GV3101 harbouring pMP90^{Gent} disarmed Ti-plasmid and binary vectors. Recombinant A. tumefaciens cultures were maintained at 100 μg/ml rifampicin, 25 μg/ml gentamicin and 25 μg/ml kanamycin. Transgenic plants were selected by spraying with a BASTA (Hoechst) herbicide solution (0.1% for one-two week old plants, 0.5% for two-three week old plants and 1% for four week old plants).

For confocal laser scanning microscopy of tissues expressing GFP under the control of the APB24 promoter, GFP fluorescence in transgenic plants was examined with a Leica TCS 4D confocal laser scanning microscope using a fluorescein isothiocyanate filter set. For analysis, fresh tissues were mounted in 5% Small DNA Low Melt Agarose (Biozym, Hessisch-Oldendorf, Germany) containing 0.005% Silwet L-77 (OSi Specialities, Slough, UK). Agarose blocks of about 0.5×0.5×1 cm were mounted on a Leitz 1512 Vibratome stage and sections of 100-150 μm were cut.

For histochemical expression analysis, portions of tissue or whole seedlings of transgenic plants, bearing APB24 promoter-GUS fusions, were stained for GUS activity according to Jefferson, et al., 1987 except that K₃Fe(CN)₆ and K₄Fe(CN)₆ were omitted from the staining solution. To detect GUS expression, plants were stained in 0.75 mg/ml 5-bromo-4-chloro-3-indoly-β-D-glucuronid acid, 0.1 M sodium phosphate buffer, pH 7.0, 20% methanol. Before examination, plant tissue was cleared in 70% ethanol.

As FIG. 8A-8F show, the expression of the very long chain fatty ω-alcohol dehydrogenase described by this invention could be localised to the protodermis and epidermis, confirming results of mRNA in situ hybridisation obtained in the first experiment of this example. It follows, that the said gene has the specific function in oxidation of ω-hydroxyfatty acids, which are required for biosynthesis of extracellular matrix polymers in the epidermis. As shown by FIGS. 8G and 8H, the activity of the said gene could be detected in accord with its function in the cells, which had to rapidly produce extracellular matrix polymers.

Furthermore, transcriptional regulatory sequences required for the epidermis-specific expression of the APB24 gene, encoding the very long chain fatty ω-alcohol dehydrogenase described by this invention, are located within the fragment as defined in SEQ ID NO:3.

### REFERENCES

Agrawal, V. P., and Kolattukudy, P. E. (1978a). Mechanism of action of a wound-induced ω-hydroxyfatty acid, NADP oxidoreductase from potato tuber Solanum tuberosum L. Archives Biochem Biophys 191, 466-478.
Agrawal, V. P., and Kolattukudy, P. E. (1978b). Purification and characterization of a wound-induced ω-hydroxy fatty acid: NADP: oxidoreductase from potato tuber disks (Solanum tuberosum L.). Archives Biochem Biophys 191, 452-465.
Araki, T., Nakatani, M., and Iwabuchi, M. (1998). ADHESION OF CALYX EDGES, a gene involved in the regulation of postgenital fusion in Arabidopsis. Paper presented at: 9th International Conference on Arabidopsis Research (University of Wisconsin, Madison).
Araki, T., and Nakatani-Goto, M. (1999). Arabidopsis ADHESION OF CALYX EDGES (ACE), genomic. Published Only in DataBase.
Ausubel et al., Short Protocols in Molecular Biology, John Wiley & Sons; 5th edition (2002), ISBN: 0471250929
Bäumlein et al., Mol. Gen. Genet. 225 (1991), 459-467
Bechtold, N., Ellis, J., and Pelletier, G. (1993). In planta Agrobacterium gene transfer by infiltration of adult Arabidopsis thaliana plants. C R Acad Sci Paris, Life Sciences 316, 1194-1199.
Benveniste, I., Tijet, N., Adas, F., Philipps, G., Salaun, J.-P., and Durst, F. (1998). CYP86A1 from Arabidopsis thaliana encodes a cytochrome P450-dependent fatty acid omega-hydroxylase. Biochem. Biophys. Res. Comm. 243, 688-693.
Bernards, M. A. (2002). Demystifying suberin. Canad. J. Bot. 80, 227-240.
Bremer, J., and Osmundsen, H. (1984). Fatty acid oxidation and its regulation. In Fatty Acid Metabolism and Its Regulation, S, Numa, ed. (Amsterdam, Elsevier Science Publishers B.V.), pp. 113-154.
Broun, P., Boddupalli, S., and Somerville, C. (1998). A bifunctional oleate 12-hydroxylase: desaturase from Lesquerella fendleri. Plant J 13, 201-210.
Broun, P., and Somerville, C. (1997). Accumulation of ricinoleic, lesquerolic, and densipolic acids in seeds of transgenic Arabidopsis plants that express a fatty acyl hydroxylase cDNA from castor bean. Plant Physiol 113, 933-942.
Browse, J., McCourt, P. J., and Somerville, C. R. (1986). Fatty acid composition of leaf lipids determined after combined digestion and fatty acid methyl ester formation from fresh tissue. Anal Biochem 152, 141-145.
Callis et al., 1987, Genes Oevel. 1, 1183-1200.
Cavener, D. R. (1992). GMC oxidoreductases. A newly defined family of homologous proteins with diverse catalytic activities. J. Mol. Biol. 223, 811-814.
Cogoni, C., and Macino, G. (2000). Post-transcriptional gene silencing across kingdoms. Curr Opin Genet Dev 10, 638-643.
Fiedler et al., Plant Mol. Biol. 22 (1993), 669-679.
Fink, G., Montgomery, J., David, F., Garneau, M., Livni, E., Elmaleh, D., Strauss, H., and
Brunengraber, H. (1990). Metabolism of beta-methyl-heptadecanoic acid in the perfused rat heart and liver. J Nucl Med 31, 1823-1830.
Gielen et al., EMBO J. 8 (1989), 23-29.
Gunstone, F. D., and Hamilton, R. J. (2001). Oleochemical Manufacture & Applications (CRC Press)
Hammond, S. M., Caudy, A. A., and Hannon, G. J. (2001). Post-transcriptional gene silencing by double-stranded RNA. Nat Rev Genet. 2, 110-119.
Haseloff, J., and Siemering, K. R. (1998). The uses of green fluorescent protein in plants. In
Green fluorescent protein: Properties, applications, and protocols., M. Chalfie, and S. Kain, eds. (Chichester, John Wiley and Sons Ltd.; Wiley-Liss, Ltd., Chichester, England), pp. 191-220.
Holloway, P. J. (1982). The chemical constitution of plant cutins. In The Plant cuticle: papers presented at an international symposium organized by the Linnean Society of London, held at
Burlington House, London, 8-11 Sep. 1980, D. F. Cutler, K. L. Alvin, and C. E. Price, eds. (London, Academic Press), pp. 45-85.
Holloway, P. J. (1983). Some variations in the composition of suberin from the cork layers of higher plants. Phytochemistry 22, 495-502.
Huijser, P., Klein, J., Loennig, W. E., Meijer, H., Saedler, H., and Sommer, H. (1992).
Bracteomania: an inflorescence anomaly is caused by the loss of function of the MADS-box gene SQUAMOSA in Antirrhinum majus. EMBO J. 11, 1239-1249.
Ichihara, K., Kusunose, E., Noda, Y., and Kusunose, M. (1986a). Some properties of the fatty alcohol oxidation system and reconstitution of microsomal oxidation activity in intestinal mucosa. Biochimica et Biophysica Acta (BBA)—Lipids and Lipid Metabolism 878, 412-418.
Ichihara, K., Noda, Y., Tanaka, C., and Kusunose, M. (1986b). Purification of aldehyde dehydrogenase reconstitutively active in fatty alcohol oxidation from rabbit intestinal microsomes. Biochimica Et Biophysica Acta 878, 419-425.
Jefferson, R. A., Kavanagh, T. A., and Bevan, M. W. (1987). GUS fusions: β-glucuronidase as a sensitive and versatile gene fusion marker in higher plants. EMBO J. 6, 3901-3908.
Krolikowski, K. A., Victor, J. L., Wagler, T. N., Lolle, S. J., and Pruitt, R. E. (2003). Isolation and characterization of the Arabidopsis organ fusion gene HOTHEAD. Plant J 35, 501-511.
Le Bouquin, R., Pinot, F., Benveniste, I., Salaun, J.-P., and Durst, F. (1999). Cloning and functional characterization of CYP94A2, a medium chain fatty acid hydroxylase from Vicia sativa. Biochem Biophys Res Comm 261, 156-162.
Le Bouquin, R., Skrabs, M., Kahn, R., Benveniste, I., Salaun, J.-P., Schreiber, L., Durst, F., and Pinot, F. (2001). CYP94A5, a new cytochrome P450 from Nicotiana tabacum is able to catalyze the oxidation of fatty acids to the ω-alcohol and to the corresponding diacid. Eur J Biochem 268, 3083-3090.
Lee, T. (1979). Characterization of fatty alcohol: NAD+ oxidoreductase from rat liver. The Journal Of Biological Chemistry 254, 2892-2896.
Leisy et al., Plant Mol. Biol. 14 (1990), 41-50.
Luehrsen, and Walbot, 1991, Mol. Gen. Genet. 225, 81-93.
Marchler-Bauer, A., Anderson, J. B., DeWeese-Scott, C., Fedorova, N. D., Geer, L. Y., He, S., Hurwitz, D. I., Jackson, J. D., Jacobs, A. R., Lanczycki, C. J., et al. (2003). CDD: a curated Entrez database of conserved domain alignments. Nucl Acids Res 31, 383-387.
Matzke, K., and Riederer, M. (1991). A comparative study into the chemical constitution of cutins and suberins from Picea abies (L.) Karst., Quercus robur L., and Fagus sylvatica L. Planta 185, 233-245.
Nelson, D. R., Koymans, L., Kamataki, T., Stegeman, J. J., Feyereisen, R., Waxman, D. J.,
Waterman, M. R., Gotoh, O., J., C. M., W., E. R., et al. (1996). P450 superfamily: Update on new sequences, gene mapping, accession numbers and nomenclature. Pharmacogenetics 6, 1-42.
Nerr et al., EMBO J. 4 (1985), 1373-1380.
Niederl, S., Kirsch, T., Riederer, M., and Schreiber, L. (1998). Co-permeability of ³H-labeled water and ¹⁴C-labeled organic acids across isolated plant cuticles. Investigating cuticular paths of diffusion and predicting cuticular transpiration. Plant Physiol 116, 117-123.
Pedersen et al., Cell 29 (1982), 1015-1026.
Pinot, F., Benveniste, I., Salaun, J.-P., Loreau, O., Noel, J.-P., Schreiber, L., and Durst, F. (1999). Production in vitro by the cytochrome P450 CYP94A1 of major C18 cutin monomers and potential messengers in plant-pathogen interactions: Enantioselectivity studies. Biochem J 342, 27-32.
Potenza, C., Aleman, L., and Sengupta-Gopalan, C. (2004). Invited review: Targeting transgene expression in research, agricultural, and environmental applications: Promoters used in plant transformation. In Vitro Cell Dev Biol-Plant 40, 1-22.
Quatroccio et al., Plant Mol. Biol. 15 (1990), 81-93).
Rethmeier, et al., 1997; Plant Journal. 12(4):895-899.
Rizzo, W. B., Craft, D. A., Dammann, A. L., and Phillips, M. W. (1987). Fatty alcohol metabolism in cultured human fibroblasts. Evidence for a fatty alcohol cycle. J. Biol. Chem. 262, 17412-17419.
Rocha-Sosa et al., EMBO J. 8 (1989), 23-29.
Rose and Beliakoff, 2000, Plant Physiol. 122 (2), 535-542.
Sambrok et al., Molecular Cloning, A Laboratory Manual, 3rd edition (2001) Cold Spring Harbour Laboratory Press, Cold Spring Harbour, NY. ISBN: 0879695773
Santrucek, J., Simanova, E., Karbulkova, J., Simkova, M., and Schreiber, L. (2004). A new technique for measurement of water permeability of stomatous cuticular membranes isolated from Hedera helix leaves. J Exp Bot 55, 1411-1422.
Schramke, V., and Allshire, R. (2004). Those interfering little RNAs! Silencing and eliminating chromatin. Curr Opin Genet Dev 14, 174-180.
Smith, M., Moon, H., and Kunst, L. (2000). Production of hydroxy fatty acids in the seeds of Arabidopsis thaliana. Biochem Soc Trans 28, 947-950.
Stockhaus et al., Proc. Natl. Acad. Sei. USA 84 (1987), 7943-7947.
Stockhaus et al., EMBO J. 8 (1989), 2445-2451.
Sumimoto, H., and Minakami, S. (1990). Oxidation of 20-hydroxyleukotriene B4 to 20-carboxyleukotriene B4 by human neutrophil microsomes. Role of aldehyde dehydrogenase and leukotriene B4 omega-hydroxylase (cytochrome P-450LTB omega) in leukotriene B4 omega-oxidation. J Biol Chem 265, 4348-4353.
Thompson et al., Nucleic Acids Research 22 (1994), 4673-4680
Vamecq, J., De Hoffmann, E., and Van Hoof, F. (1985). The microsomal dicarboxylyl coenzyme A. Biochem. J. 230, 683-694.
Vanhanen, S., West, M., Kroon, J. T. M., Lindner, N. I., Casey, J., Cheng, Q., Elborough, K. M., and Slabas, A. R. (2000). A consensus sequence for long-chain fatty-acid alcohol oxidases from Candida identifies a family of genes involved in lipid omega-oxidation in yeast with homologues in plants and bacteria. J. Biol. Chem. 275, 4445-4452.
Vasil et al., 1989, Plant Physiol. 91, 1575-1579.
Vogg, G., Fischer, S., Leide, J., Emmanuel, E., Jetter, R., Levy, A. A., and Riederer, M. (2004). Tomato fruit cuticular waxes and their effects on transpiration barrier properties: functional characterization of a mutant deficient in a very-long-chain fatty acid β-ketoacyl-CoA synthase. J Exp Bot 55, 1401-1410.
Wellesen, K., Durst, F., Pinot, F., Benveniste, I., Nettesheim, K., Wisman, E., Steiner-Lange, S., Saedler, H., and Yephremov, A. (2001). Functional analysis of the LACERATA gene of Arabidopsis provides evidence for different roles of fatty acid omega-hydroxylation in development. Proc Natl Acadl Scil USA 98, 9694-9699.
Werck-Reichhart, D., Bak, S., and Paquette, S., eds. (2001). Cytochromes P450 (http://www.aspb.org/publications/arabidopsis).
Wheelan, P., Hankin, J. A., Bilir, B., Guenette, D., and Murphy, R. C. (1999). Metabolic Transformations of Leukotriene B4 in Primary Cultures of Human Hepatocytes. J Pharmacol Exp Ther 288, 326-334.
XU et al., 2003, Science in China Series C, Vol. 46 No. 6, 561-569).
Yoshihara et al., FEBS Lett. 383 (1996), 213-218).
Zheng et al., Plant J. 4 (1993), 357-366.

1. A method for the generation of a transgenic plant having a changed lipid composition and/or modified extracellular matrix polymers as compared to a corresponding non-transgenic wild type plant, the method comprising the step of stably integrating at least one deoxyribonucleic acid into the genome of a plant cell, plant tissue, protoplast or plant propagation material by transformation of said plant cell, plant tissue, protoplast or plant propagation material and regeneration of the obtained plant cell or plant tissue to a transgenic plant, wherein said at least one deoxyribonucleic acid is:
(a) a deoxyribonucleic acid molecule encoding at least one antisense RNA effecting a decreased expression of at least one endogenous gene, said at least one endogenous gene encoding a protein including an amino acid sequence according to SEQ ID NO:2,
(b) deoxyribonucleic acid molecules effecting a decreased expression of at least one endogenous gene by co-suppression, said at least one endogenous gene encoding a protein including an amino acid sequence according to SEQ ID NO:2,
(c) a deoxyribonucleic acid molecule encoding at least one ribozyme, said ribozyme specifically cleaving the transcripts of at least one endogenous gene encoding a protein including an amino acid sequence according to SEQ ID NO:2,
(d) deoxyribonucleic acid molecules encoding at least one antisense RNA and at least one sense RNA, wherein said at least one antisense RNA and said at least one sense RNA form a double stranded RNA molecule which effects a decreased expression of at least one endogenous gene encoding a protein including an amino acid sequence according to SEQ ID NO:2,
(e) a deoxyribonucleic acid molecule encoding an antibody, wherein said antibody effects a decreased expression of at least one endogenous gene encoding a protein including an amino acid sequence according to SEQ ID NO:2 by binding to said protein,
(f) a deoxyribonucleic acid molecule comprising at least one transposable element, wherein said transposable element integrates into at least one endogenous gene encoding a protein including an amino acid sequence according to SEQ ID NO:2, said integration effecting a decreased expression of said at least one endogenous gene or results in the synthesis of inactive protein, or
(g) a deoxyribonucleic acid molecule comprising at least one T-DNA, wherein said T-DNA integrates into at least one endogenous gene encoding a protein including an amino acid sequence according to SEQ ID NO:2, said integration effecting a decreased expression of said at least one endogenous gene or results in the synthesis of inactive protein.

2. The method of claim 1, wherein the at least one deoxyribonucleic acid (a), (b), (d), (f) or (g) comprises the nucleotide sequence according to SEQ ID NO: 1.

3. The method according to claim 1 or 2, comprising the step of stably integrating at least one deoxyribonucleic acid comprising the nucleotide sequence according to SEQ ID NO: 1, or a fragment thereof, or of a nucleic acid hybridizing to the nucleic sequence according to SEQ ID NO:1 under stringent conditions, or of a nucleic acid encoding a protein including an amino acid sequence according to SEQ ID NO:2, or a fragment thereof, wherein said at least one deoxyribonucleic acid or said fragment thereof, or said nucleic acid hybridizing to the nucleic sequence, or said nucleic acid encoding a protein including an amino acid sequence according to SEQ ID NO:2, or a fragment thereof, is expressed in antisense or sense-orientation in respect to the endogenous sequence responsible for the changed lipid composition, into the genome of a plant cell, plant tissue, protoplast or plant propagation material by transformation of said plant cell, plant tissue, protoplast or plant propagation material and regeneration of the obtained plant cell or plant tissue to a transgenic plant.

4. The method according to claim 3, wherein the expression of said at least one deoxyribonucleic acid comprising the nucleotide sequence according to SEQ ID NO:1, or said fragment thereof, or said nucleic acid hybridizing to the nucleic sequence according to SEQ ID NO:1 under stringent conditions, or of said nucleic acid encoding a protein including an amino acid sequence according to SEQ ID NO:2, or a fragment thereof, is controlled by a tissue or organ specific promoter.

5. The method according to claim 4, wherein the promoter includes the napin promoter from Brassica napus or from Arabidopsis thaliana, or the hydroxylase promoter from Lesquerella fendleri.

6. The method according to claim 1, wherein the plant cell, plant tissue, protoplast or plant propagation material is derived from a monocotyledonous or dicotyledonous plant.

7. A transgenic plant, obtainable by the method according to claim 1.

8. A transgenic plant, plant cell, plant tissue, protoplast or plant propagation material transformed with an expressible deoxyribonucleic acid comprising the nucleotide sequence according to SEQ ID NO:1, or a fragment thereof, or a nucleic acid hybridizing to the nucleic sequence according to SEQ ID NO:1 under stringent conditions, or of a nucleic acid encoding a protein having including an amino acid sequence according to SEQ ID NO:2, or a fragment thereof, wherein the expressible deoxyribonucleic acid is stably integrated into the genome of the plant, plant cell, plant tissue, protoplast or plant propagation material.

9. The transgenic plant, plant cell, plant tissue, protoplast or plant propagation material according to claim 8, which is regenerable to a seed forming plant.

10. A seed or a fruit transgenic in a nucleic acid comprising the nucleotide sequence according to SEQ ID NO:1, or a fragment thereof, or a nucleic acid hybridizing to the nucleic sequence according to SEQ ID NO:1 under stringent conditions, or a nucleic acid encoding a protein including an amino acid sequence according to SEQ ID NO:2, or a fragment thereof, obtainable from a plant according to any of claims 7 to 9.

11. A method for the production of α-,ω-dicarboxylic fatty acids comprising using a transgenic plant according to any of claims 7 to 9.

12. Recombinant nucleic acid molecule comprising a promoter, which initiates transcription in plant cells, and at least one nucleic acid sequence including:
a) SEQ ID NO:1;
b) nucleic acid hybridising to the nucleic sequence according to SEQ ID NO:1 under stringent conditions; or
c) nucleic acid sequences encoding a protein including an amino acid sequence as identified under SEQ ID NO:2.

13. Recombinant nucleic acid molecule comprising a promoter, which initiates transcription in plant cells, and at least one nucleic acid sequence including:
a) SEQ ID NO:1;
b) nucleic acid hybridizing to the nucleic sequence according to SEQ ID NO:1 under stringent conditions; or
c) nucleic acid sequences encoding a protein including an amino acid sequence as identified under SEQ ID NO:2.

14. The method of claim 6, wherein the monocotyledonous or dicotyledonous plant is rapeseed, canola, tomato, potato, safflower, sunflower, soybean, peanut, cotton, palm, maize, sorghum, rice, barley or wheat.

15. The method of claim 11, wherein the α-,ω-dicarboxylic fatty acids are α-,ω-dicarboxylic fatty acids bearing an omega-aldehyde or omega-carboxyl group.


SEQUENCE LISTING (7 sequences)

SEQ ID NO: 1 - DNA, 1785 bp, Arabidopsis thaliana
atggctctcaagctctttctctttgctcttcttctctgtctcccgacttctctctcctccactgcctctaaaggtaaagagaagaagtcgaagtttaatccatacagatacacattcatcgataaagcaagcacattctcatcatcttcctcatcttcattttcatccaacggtcaagattcgtcctacgactacatagtcatcggaggtggaaccgcagggtgtcctctcgccgcaacgttgtcgcagaatttcagcgttcttgttttagagagaggtggcgttccgtttacaaacgcaaacgtttctttcctcaggaattttcacatcggacttgctgacatttcagcttcttccgcgtctcaagcgtttgtttccactgacggcgtttacaacgcccgtgctagagttctcggtggcggttcctgtattaacgccggtttttactccagagccgatgctgcgttcgtgaagcgagcaggatgggatccgaagctggtgaaggagtcgtatccatgggtggagagagagattgttcatcagccaaagttaacgttatggcagaaagctctcagagacagtcttttagaggttggagtcagacctttcaatggtttcacttacgatcacgtttccggaaccaaaatcggcggtacaattttcgacagattcggccgtcgtcacaccgcggcggagcttctcgcttacgctaatcctcagaagcttagagtcttgatctacgccaccgtgcaaaaaatcgtctttgacacttctggaacaaggcctcgagtaacaggagtaatattcaaagatgagaaaggtaatcaacaccaggctttactctcgaatagaaagggaagtgaagtgatcttatctagtggagctattgggtcaccacagatgctgatgttaagtgggattggacctaagaaggagcttcagaggctgaagattcctgtggttttagagaatgagcatgtaggaaaaggaatggctgataatcccatgaacacgatcttggtgccttcaaaggcgcctatagagcagtcacttattcagactgttggaattacaaagatgggtgtgtatgttgaagccagtactggctttgggcaatctcctgagagtattcatactcactatgggattatgtcgaacaagaatgaattgttttccaccattcctgcaaagcagagaagaccagaagcaacgcaagcttacatcacaagaaacaaataccaacttcacgaagcattcaatggaagtttcatcttggagaaacttgcttacccgatctctagagggcatttgagcttggtcaacacaaatgttgatgacaacccttcagtcaccttcaattactttaaacacccggtggatctccaacgctgtgttgaagccattcgtcttgtttccaaagtggtgacgtctaaccgtttcttaaactacacgcagtgtgacaagcaaaacgtacacaagatgcttagcttaagcgtcaaggcaaacatcaatctaaggccaaagcaactgaacgataccaaatcaatggctcagttctgcaaagacactgttgtcacaatctggcactaccatggtggatgtcttgtgggtaaagttgtgagccctaaccgcaaagttcttggtgtcgacaggctcagagttattgatggttcaacgtttgacgagtctccaggaaccaacccgcaagctactatgatgatgatgggaagatacatgggagtcaagattcttcgggagagacttggaaacaaagctggtgtttag

SEQ ID NO: 2 - protein, 594 aa, Arabidopsis thaliana
MALKLFLFALLLCLPTSLSSTASKGKEKKSKFNPYRYTFIDKASTFSSSSSSSFSSNGQDSSYDYIVIGGGTAGCPLAATLSQNFSVLVLERGGVPFTNANVSFLRNFHIGLADISASSASQAFVSTDGVYNARARVLGGGSCINAGFYSRADAAFVKRAGWDPKLVKESYPWVEREIVHQPKLTLWQKALRDSLLEVGVRPFNGFTYDHVSGTKIGGTIFDRFGRRHTAAELLAYANPQKLRVLIYATVQKIVFDTSGTRPRVTGVIFKDEKGNQHQALLSNRKGSEVILSSGAIGSPQMLMLSGIGPKKELQRLKIPVVLENEHVGKGMADNPMNTILVPSKAPIEQSLIQTVGITKMGVYVEASTGFGQSPESIHTHYGIMSNKNELFSTIPAKQRRPEATQAYITRNKYQLHEAFNGSFILEKLAYPISRGHLSLVNTNVDDNPSVTFNYFKHPVDLQRCVEAIRLVSKVVTSKRFLNYTQCDKQNVHKMLSLSVKANINLRPKQLNDTKSMAQFCKDTVVTIWHYHGGCLVGKVVSPNRKVLGVDRLRVIDGSTFDESPGTNPQATMMMMGRYMGVKILRERLGNKAGV

SEQ ID NO: 3 - DNA, 1942 bp, Arabidopsis thaliana
caaaagaggactaaagaggagagaaacaaagaatcttcttactctccaaaggtttgatctccagttcttgacgtttttttctccttattccctcactcatgagtccaaaacgctgcgtttatagggaccttttgctctgttttattagaacttctcattatacacacgagtgaattagaacttgaaaacgttgtggcatcatttgaatatttttccttcaaataaacaaaaaaaaaaagatgaagatgaaaaccaataataaatatatgtacgttctataataactcgtttcatgaaagtggaagcatgggatgagatgccaatcccatgaaaagtgagcaaaaattaatatttacattaactgctgagtgggtaaagtttaataaccattttctaaaaataacttttattcagaatccaactcatggattggtgtgtgtatttattatcatcatttctatacacatatccacccccaataactctgtcaattatacacctcaaaatcactgtaagttataaaaaaatgaatttctacaattaatcaaagaccgtccaaagccttcgtggctaattggtgttaaactattcacatttttagtcaagtatttttgcatattatatgagattaattttgccacacaagatcgttaataaactcgacatgatgattctgtttatgtggcttctattaactaggaaatgaaacaatgtagttatagttgtgactgatatgaagtcagagaaaatggctacgtaatcttatatgaccagaccaacgtacgtgatgcgatgtgacatttttcattcaacactaaagttaaaaccttgattgtttttgtattacatttggcaactaatccaatatgttttgttagttgttagtactaagtactgactgatgatcacattcacgctcaaccaaataaaacggatgatattgtttttggttaggtgaaagaacaattaaattagttgccaaaaacataacccgtgaagatttatgttagtaaccaacctacagtcataattaataagtttatgactaatagtaattaaatgacttgccaaaaacatggctctgtatagtatagtttttagattttggttaggtgaagatgattaattgatacactatgaatctatgaagacttgatccaacggctaccagatctccgacttcaactgttttcatatcgccattaatgatacagacaagctgtactactatcctttttatctctttattgttttatatgattttacagtaatactattactaagtactaacaaattgtttttctttgcagctggtcccaaaagtcaaaatcatgggacaaaaaaacgagttattattcttattatcatcaccaaggtaaaaaaatgtgaaaggaaatccttttaaggcttagtttcaatataaatacaaagatgttatttgatagtgattgttctttgtaacgaagtacttatcattaatttcaatttgcaacaaacaattctgtaacaataagtctaatgtgaggaaagttactagctggtaaaataaattagtaggtaactattttttatgagtaaatgagggaactattaattgagtaagatgattcatatgtccttttcatttgctatattcgacagaatgggacacacctaaaatcaaatttacgttttagtgtagaccaccccaacatctctttattacacaacttgtgtttttttttactatatgatttttttaccgaatgttgttgtctaaccaaaattcaatgtaccgtataagactataagttatacaactcgtagaaatggttacatatatgtaacaatatatagagacacatgtgggtgtaattaatatagttgctctgttggatcgttagtgttatttataggagaagaaaacacacaaaacaagaccattcatcaaaagccacataag

SEQ ID NO: 4 - DNA, 57 bp, Artificial sequence
  Primer
ctcgagtttaggtgacactatagaactggagcctccactgcctctaaaggtaaagag

SEQ ID NO: 5 - DNA, 52 bp, Artificial sequence
  Primer
ctcgagtaatacgactcactatagggagacaccaagaactttgcggttaggg

SEQ ID NO: 6 - DNA, 38 bp, Artificial sequence
  Primer
aaaaaaagcttcaaaagaggactaaagaggagagaaac

SEQ ID NO: 7 - DNA, 37 bp, Artificial sequence
  Primer
aaaaaatctagacttatgtggcttttgatgaatggtc